(19) United States
(12) Patent Application Publication
(10) Pub. No.: US 2021/0220438 A1
(43) Pub. Date: Jul. 22, 2021

(54) BIOMARKER PANEL FOR MONITORING KIDNEY HEALTH

(71) Applicant: The Regents of the University of California, Oakland, CA (US)

(72) Inventors: Joachim Ix, La Jolla, CA (US); Michael Shlipak, San Francisco, CA (US)

(21) Appl. No.: 17/154,824

(22) Filed: Jan. 21, 2021

Related U.S. Application Data
(60) Provisional application No. 62/964,520, filed on Jan. 22, 2020.

(51) Int. Cl. A61K 38/17; A61P 13/12; G01N 33/68; A61K 38/20; A61K 45/06

(52) U.S. Cl. CPC A61K 38/1722; A61P 13/12; G01N 33/68; G01N 2800/52; A61K 38/1741; A61K 45/06; A61K 38/20

(57) ABSTRACT

The present disclosure provides a panel of biomarkers for use in diagnosing and/or monitoring kidney health during a therapeutic treatment for a therapeutic regimen that induces renal impairment or chronic kidney disease. Such monitoring may be useful in subjects undergoing treatment a disease such as diabetes mellitus and/or hypertension, where a therapeutic regimen results in increased levels of blood creatinine.

### CROSS-REFERENCE TO RELATED APPLICATIONS

This application claims priority to U.S. Provisional Application No. 62/964,520, filed Jan. 22, 2020, the entire content of which is incorporated herein by reference.

### STATEMENT OF GOVERNMENT SUPPORT

This disclosure was made with government support under Grant No. DK098234, awarded by the National Institutes of Health. The United States government has certain rights in the invention.

### TECHNICAL FIELD

This disclosure relates generally to kidney health and more specifically to methods for monitoring kidney health in a subject undergoing treatment with a therapeutic regimen that causes renal impairment or chronic kidney disease.

### BACKGROUND

Random assignment to the intensive systolic blood pressure (SBP) arm (<120 mm Hg) in the Systolic Blood Pressure Intervention Trial (SPRINT) results in more rapid declines in estimated glomerular filtration rates (eGFRs) than in the standard arm (SBP <140 mm Hg). Intensive BP lowering results in higher blood creatinine, which is typically indicative of decreased kidney function, thereby causing physicians concern that a patient is suffering from kidney damage. However, an increase in blood creatinine levels may also be due to changes in blood flow, a hemodynamic effect that is benign to the patient. However, a longitudinal subgroup analysis of the SPRINT clinical trial participants with prevalent chronic kidney disease (CKD) defined as eGFR <60 mL/min/1.73 m² by the CKD-EPI (CKD Epidemiology Collaboration) creatinine-cystatin C equation at baseline showed that the change in eGFR reflects hemodynamic effects rather than accelerated intrinsic kidney damage.

Furthermore, sodium glucose transporter 2 (SGLT2) inhibitors are a relatively new class of drugs for treating type 2 diabetes, which have been shown to result in lower risk for progression to dialysis in long-term follow-up. However, when patients first begin a therapeutic regimen of SGLT2 inhibitors, they typically experience an acute change in blood flow to the kidney, which results in a rise in serum creatinine. This causes concerns to practitioners that the drug may be harming the kidneys, rather than being beneficial long-term. While some patients may indeed experience intrinsic kidney damage due to marked reductions in blood flow, resulting in cessation of SGLT2 inhibitor therapy and the benefit associated therewith, there is currently no way to differentiate between these two patterns of creatinine change.

Thus, a need exists for kidney health biomarkers that can differentiate intrinsic kidney damage from hemodynamic changes in patients taking therapeutics that lower eGFR and result in higher blood creatinine. The present disclosure addresses this need.

### SUMMARY OF THE DISCLOSURE

The present disclosure is based on the identification of a panel of biomarkers that can differentiate between hemodynamic changes in serum creatinine from intrinsic kidney damage in patients taking various therapeutics, including but not limited to therapeutics for treating diabetes mellitus and hypertension. Accordingly, the present disclosure provides methods for monitoring kidney health in a subject undergoing treatment for various diseases comprising diabetes mellitus, hypertension, or any disease that affects kidney health, or induced renal impairment or chronic kidney disease. In particular, the present disclosure provides a combination of one or more of eight (8) biomarkers that can be used in conjunction with eGFRs and albumin-creatinine ratio (ACR) to monitor kidney health by assessing intrinsic versus hemodynamic changes in kidney function in a subject undergoing treatment with various therapeutics that influence renal function. The methods include routinely measuring the levels of the disclosed novel biomarkers in conjunction with eGFR and ACR, and determining whether the therapeutic treatment should be discontinued.

In one aspect, the present disclosure provides a method for treating diabetes mellitus in a subject in need thereof comprising, or alternatively consisting essentially of, or further yet consisting of: (a) administering a therapeutic regimen to the subject after the levels of one or more of α₁-microglobulin (A1M), β₂-microglobulin (B2M), kidney injury molecule 1 (KIM-1), interleukin 18 (IL-18), monocyte chemoattractant protein 1 (MCP-1), neutrophil gelatinase-associated lipocalin (NGAL), uromodulin (UMOD), or human cartilage glycoprotein 39 (YKL-40) were measured in a first biological sample isolated from the subject; and (b) comparing the measured levels of the one or more of A1M, B2M, KIM-1, IL-18, MCP-1, NGAL, UMOD, or YKL-40 in a second biological sample from the subject to the levels of the first biological sample.

In some embodiments, the levels of one or more comprise the levels of KIM-1, IL-18, MCP-1, NGAL, UMOD, and YKL-40. In some embodiments, the levels of one or more comprises the levels of a combination of KIM-1, NGAL and UMOD; MCP-1, IL-18 and YKL-40; UMOD, MCP-1, and IL-18; NGAL, MCP-1, and IL-18; or KIM-1, MCP-1 and IL-18. In some embodiments, the measured levels of one or more comprises: (1) the measured levels of at least one of UMOD, NGAL, KIM-1, and (2) the measured levels of at least one of MCP-1, IL-18 or YKL-40.

In some embodiments, the method for treating diabetes mellitus in a subject in need thereof further comprises administering the therapeutic regimen to the subject if the one or more measured levels of KIM-1, IL-18, MCP-1, NGAL, UMOD, or YKL-40 in the second biological sample are not elevated when compared to the measured levels of the first biological sample. In some embodiments, a lack of elevated levels of the one or more of KIM-1, IL-18, MCP-1, NGAL, UMOD, or YKL-40 in the second biological sample as compared to the measured levels of the first biological sample is indicative of continued kidney health. In some embodiments, a lack of the elevated levels of the one or more of KIM-1, IL-18, MCP-1, NGAL, UMOD, or YKL-40 in the second biological sample as compared to the measured levels of the first biological sample indicates that the therapeutic regimen should be continued. In some embodiments, the method further comprises repeating step (a)-(b) during treatment. In some embodiments, step (a)-(b) are repeated at a predetermined time selected from 1 month, 3 months, 6 months, 9 months, 1 year, 2 years, or 3 years. In some embodiments, the first and second biological samples are blood and/or urine.

In some embodiments, the therapeutic regimen comprises, or consists essentially of, or yet further consists of administering a therapeutic selected from sodium glucose transporter 2 (SGLT2) inhibitors, angiotensin-converting enzymes inhibitors, nonsteroidal anti-inflammatory medications, antihypertensive medications, intensive blood pressure lowering medications or a combination thereof. In some embodiments, the therapeutic regimen comprises administering at least one sodium glucose transporter 2 (SGLT2) inhibitor selected from canagliflozin, dapagliflozin, or empagliflozin.

In some embodiments, the therapeutic regimen comprises, or consists essentially of, or further consists of administering at least one sodium glucose transporter 2 (SGLT2) inhibitor selected from canagliflozin, dapagliflozin, or empagliflozin, and further comprises administering a non-SGLT2 inhibitor therapeutic regimen. In some embodiments, the non-SGLT2 inhibitor therapeutic for the treatment of diabetes mellitus is selected from metformin, sulphonylureas, nateglinide, repaglinide, thiazolidinediones, pioglitazone PPARα-glucosidase inhibitors, insulin and insulin analogues, Glucagon-like peptide 1 (GLP-1) and GLP-1 analogues or dipeptidyl peptidase-4 (DPP-4) inhibitors.

In some embodiments, therapeutic regimen comprises, or consists essentially of, or yet consists of administering an intensive blood pressure lowering therapy. In some embodiments, the intensive blood pressure lowering therapy is an antihypertensive regimen selected from diuretics, renin-angiotensin system (RAS) antagonists, β-adrenergic blockers, α-adrenergic blockers, calcium channel blockers, or a combination thereof. In some embodiments, the antihypertensive regimen is selected from chlorthalidone, chlorothiazide, hydrochlorothiazide, indapamide, and metolazone, furosemide, bumetanide, amlodipine, Azilsartan, or acebutolol.

In some embodiments, the subject is at risk of an adverse health condition when the estimated glomerular filtration rates (eGFR) of the subject is less than 60 ml/min/1.73 m². In some embodiments, the eGFR is measured in the first and second biological samples. In some embodiments, the therapeutic regiment is discontinued if the levels of the one or more of KIM-1, IL-18, MCP-1, NGAL, UMOD, or YKL-40 in the second biological sample are elevated when compared to the measured levels of the first biological sample in conjunction with a rapid loss of eGFR, A1M, or B 1M. In some embodiments, the loss of eGFR is at least 11% reduction, or eGFR in the second biological sample is less than 40 ml/min/1.73m². In some embodiments, the subject is at risk of renal impairment or chronic kidney disease.

In some embodiments, the method for treating diabetes mellitus in a subject in need thereof further comprises discontinuing the therapeutic regiment if the levels of the one or more of KIM-1, IL-18, MCP-1, NGAL, UMOD, or YKL-40 in the second biological sample are elevated when compared to the first biological sample and the eGFR is reduced by at least 11%, or the eGFR in the second biological sample is less than 40 ml/min/1.73 m².

In one aspect, the present disclosure provides a method of treating hypertension in a subject at risk of chronic kidney disease comprising, or consisting essentially of, or yet consisting of: (a) administering a therapeutic regimen to the subject after the levels of one or more of α₁-microglobulin (A1M), β₂-microglobulin (B2M), kidney injury molecule 1 (KIM-1), interleukin 18 (IL-18), monocyte chemoattractant protein 1 (MCP-1), neutrophil gelatinase-associated lipocalin (NGAL), uromodulin (UMOD), or human cartilage glycoprotein 39 (YKL-40) were measured in a first biological sample isolated from the subject; and (b) comparing the levels of A1M, B2M, KIM-1, IL-18, MCP-1, NGAL, UMOD, or YKL-40 in a second biological sample from the subject to the levels of the first biological sample. In some embodiments, the method further comprises repeating step (a)-(b) during treatment.

In some embodiments, the levels of one or more comprises the levels of KIM-1, IL-18, MCP-1, NGAL, UMOD, and YKL-40. In some embodiments, the levels of one or more comprises the levels of a combination of KIM-1, NGAL and UMOD; MCP-1, IL-18 and YKL-40; UMOD, MCP-1, and IL-18; NGAL, MCP-1, and IL-18; or KIM-1, MCP-1 and IL-18. In some embodiments, the measured levels of one or more comprises: (1) the measured levels of at least one of UMOD, NGAL, KIM-1, and (2) the measured levels of at least one of MCP-1, IL-18 or YKL-40.

In some embodiments, the method of treating hypertension in a subject at risk of chronic kidney disease further comprises, or consists essentially of, or yet further consists of administering the therapeutic regimen to the subject if the levels of one or more of KIM-1, IL-18, MCP-1, NGAL, UMOD, or YKL-40 in the second biological sample are not elevated when compared to the first biological sample. In some embodiments, a lack of elevated levels of the one or more KIM-1, IL-18, MCP-1, NGAL, UMOD, or YKL-40 in the second biological sample as compared to the first biological sample is indicative of continued kidney health and the therapeutic regimen should be continued. In some embodiments, the first and second biological samples are blood and/or urine.

In some embodiments, the therapeutic regimen is an antihypertensive regimen selected from diuretics, renin-angiotensin system (RAS) antagonists, β-adrenergic blockers, α-adrenergic blockers, calcium channel blockers, or a combination thereof. In some embodiments, the antihypertensive regimen is selected from chlorthalidone, chlorothiazide, hydrochlorothiazide, indapamide, and metolazone, furosemide, bumetanide, amlodipine, azilsartan, or acebutolol.

In some embodiments, the therapeutic regiment is discontinued if the one or more measured levels of KIM-1, IL-18, MCP-1, NGAL, UMOD, or YKL-40 in the second biological sample are elevated when compared to the measured levels of the first biological sample in conjunction with a rapid loss of eGFR, A1M, or B2M. In some embodiments, the loss of eGFR is at least 11% reduction, or eGFR in the second biological sample is less than 40 ml/min/1.73 m².

In one aspect, the present disclosure provides a kit or article of manufacture comprising, consisting essentially of, or further yet consisting of: (i) reagents specific to measure one or more levels of α₁-microglobulin (A1M), β₂-microglobulin (B2M), kidney injury molecule 1 (KIM-1), interleukin 18 (IL-18), monocyte chemoattractant protein 1 (MCP-1), neutrophil gelatinase-associated lipocalin (NGAL), uromodulin (UMOD), or human cartilage glycoprotein 39 (YKL-40) in a biological sample from a subject; and (ii) instructions for monitoring kidney health in the subject undergoing a therapeutic treatment that induces renal impairment or chronic kidney disease.

### BRIEF DESCRIPTION OF THE DRAWINGS

FIG. 1 shows a bar graph illustrating the percentage 1-year change between intensive (SBP target<120 mmHg) versus standard SBP target<140 mmHg) blood pressure control for estimated glomerular filtration rate, albuminuria, and urinary tubular markers in participants with chronic kidney disease among 978 patients participating in the SPRINT (Systolic Blood Pressure Intervention Trial) with CKD at baseline. Biomarkers that are small molecules that are filtered at the glomerulus were all decreased in the intensive vs. the standard arm (bars 2-4). In contrast, biomarkers that are produced within kidney tissue and found in higher concentrations in the urine in response to kidney tubule injury, repair, or inflammation were all similar in the intensive vs. the standard arm (bars 5-10). Collectively, the data suggest that changes in eGFR in response to intensive SBP lowering are predominantly driven by hemodynamic changes. Abbreviations: A1M, α₁-microglobulin; ACR, albumin-creatinine ratio; B2M, β₂-microglobulin; eGFR, estimated glomerular filtration rate; KIM-1, kidney injury molecule 1; IL-18, interleukin 18; MCP-1, monocyte chemoattractant protein 1; NGAL, neutrophil gelatinase-associated lipocalin; UMOD, uromodulin; YKL-40, human cartilage glycoprotein 39.

FIG. 2 shows a schematic of a proposed model for comprehensively assessing kidney health, and shows that a comprehensive kidney health biomarker panel would capture not only glomerular function (eGFR) and injury (albuminuria), but also kidney tubule function and injury concurrently.

### DETAILED DESCRIPTION

The present disclosure is based on the finding that a panel of biomarkers can differentiate between hemodynamic changes in serum creatinine from intrinsic kidney damage in patients undergoing treatment with various therapeutics that influence renal function. Large-scale phase 3 clinical trials have demonstrated that SGLT2 inhibitors have substantial benefit for the prevention of both cardiovascular disease (CVD) and dialysis. However, the acute changes in serum creatinine may result in many patients having to come off these life-saving medications because of the correlation between a rise in serum creatinine and intrinsic kidney damage. The present disclosure provides a panel of biomarkers for monitoring kidney health in patients undergoing treatment, for improving treatment safety, and determining whether continuing treatment would be acceptable despite changes in serum creatinine.

Before the present compositions and methods are described, it is to be understood that this disclosure is not limited to particular compositions, methods, and experimental conditions described, as such compositions, methods, and conditions may vary. It is also to be understood that the terminology used herein is for purposes of describing particular embodiments only, and is not intended to be limiting, since the scope of the present disclosure will be limited only in the appended claims.

Embodiments according to the present disclosure are described more fully hereinafter. Aspects of the present disclosure may, however, be embodied in different forms and should not be construed as limited to the embodiments set forth herein. Rather, these embodiments are provided so that the present disclosure will be thorough and complete, and will fully convey the scope of the disclosure to those skilled in the art. The terminology used in the description herein is for the purpose of describing particular embodiments only and is not intended to be limiting. Throughout and within the present disclosure various technical and patent publications are references by a citation or an Arabic numeral. The full bibliographic citations for each reference identified by an Arabic numeral is found in the reference section, immediately preceding the claims.

It is to be appreciated that certain aspects, modes, embodiments, variations and features of the present methods are described below in various levels of detail in order to provide a substantial understanding of the present technology. The definitions of certain terms as used in the specification are provided below. Unless otherwise defined, all terms (including technical and scientific terms) used herein have the same meaning as commonly understood by one of ordinary skill in the art to which this disclosure belongs. It will be further understood that terms, such as those defined in commonly used dictionaries, should be interpreted as having a meaning that is consistent with their meaning in the context of the present application and relevant art and should not be interpreted in an idealized or overly formal sense unless expressly so defined herein. While not explicitly defined below, such terms should be interpreted according to their common meaning. Unless explicitly indicated otherwise, all specified embodiments, features, and terms intend to include both the recited embodiment, feature, or term and biological equivalents thereof.

The terminology used in the description herein is for the purpose of describing particular embodiments only and is not intended to be limiting of the disclosure. All publications, patent applications, patents and other references mentioned herein are incorporated by reference in their entirety.

Unless the context indicates otherwise, it is specifically intended that the various features of the disclosure described herein can be used in any combination. Moreover, the present disclosure also contemplates that in some embodiments any feature or combination of features set forth herein can be excluded or omitted. To illustrate, if the specification states that a complex comprises components A, B and C, it is specifically intended that any of A, B or C, or a combination thereof, can be omitted and disclaimed singularly or in any combination. The term consisting of intends the recited elements and any additional elements that do not materially change of the function of the recited element or elements.

The practice of the present technology employs, unless otherwise indicated, conventional techniques of tissue culture, immunology, molecular biology, microbiology, cell biology, and recombinant DNA, which are within the skill of the art. See, e.g., Green and Sambrook eds. (2012) Molecular Cloning: A Laboratory Manual, 4th edition; the series Ausubel et al. eds. (2015) Current Protocols in Molecular Biology; the series Methods in Enzymology (Academic Press, Inc., N.Y.); MacPherson et al. (2015) PCR 1: A Practical Approach (IRL Press at Oxford University Press); MacPherson et al. (1995) PCR 2: A Practical Approach; McPherson et al. (2006) PCR: The Basics (Garland Science); Harlow and Lane eds. (1999) Antibodies, A Laboratory Manual; Greenfield ed. (2014) Antibodies, A Laboratory Manual; Freshney (2010) Culture of Animal Cells: A Manual of Basic Technique, 6th edition; Gait ed. (1984) Oligonucleotide Synthesis; U.S. Pat. No. 4,683,195; Hames and Higgins eds. (1984) Nucleic Acid Hybridization; Anderson (1999) Nucleic Acid Hybridization; Herdewijn ed. (2005) Oligonucleotide Synthesis: Methods and Applications; Hames and Higgins eds. (1984) Transcription and Translation; Buzdin and Lukyanov ed. (2007) Nucleic Acids Hybridization: Modern Applications; Immobilized Cells and Enzymes (IRL Press (1986)); Grandi ed. (2007) In Vitro Transcription and Translation Protocols, 2nd edition; Guisan ed. (2006) Immobilization of Enzymes and Cells; Perbal (1988) A Practical Guide to Molecular Cloning, 2nd edition; Miller and Calos eds, (1987) Gene Transfer Vectors for Mammalian Cells (Cold Spring Harbor Laboratory); Makrides ed. (2003) Gene Transfer and Expression in Mammalian Cells; Mayer and Walker eds. (1987) Immunochemical Methods in Cell and Molecular Biology (Academic Press, London); Lundblad and Macdonald eds. (2010) Handbook of Biochemistry and Molecular Biology, 4th edition; Herzenberg et al. eds (1996) Weir's Handbook of Experimental Immunology, 5th edition; and/or more recent editions thereof.

### Definitions

All numerical designations, e.g., pH, temperature, time, concentration, and molecular weight, including ranges, are approximations which are varied (+) or (−) by increments of 1.0 or 0.1, as appropriate, or alternatively by a variation of +/−15%, or alternatively 10%, or alternatively 5%, or alternatively 2%. It is to be understood, although not always explicitly stated, that all numerical designations are preceded by the term “about”. It also is to be understood, although not always explicitly stated, that the reagents described herein are merely exemplary and that equivalents of such are known in the art.

As used herein, the singular forms “a”, “an”, and “the” include plural references unless the context clearly dictates otherwise. Thus, for example, references to “the method” includes one or more methods, and/or steps of the type described herein which will become apparent to those persons skilled in the art upon reading the present disclosure and so forth.

As used herein, the term “about,” when referring to a measurable value such as an amount or concentration and the like, is meant to encompass variations of 20%, 10%, 5%, 1%, 0.5%, or even 0.1% of the specified amount.

As used herein, the terms “acceptable,” “effective,” or “sufficient” when used to describe the selection of any components, ranges, dose forms, etc. disclosed herein intend that said component, range, dose form, etc. is suitable for the disclosed purpose.

It is intended that reference to a range of numbers disclosed herein (for example 1 to 10) also incorporates reference to all related numbers within that range (for example, 1, 1.1, 2, 3, 3.9, 4, 5, 6, 6.5, 7, 8, 9 and 10) and also any range of rational numbers within that range (for example 2 to 8, 1.5 to 5.5 and 3.1 to 4.7) and, therefore, all sub-ranges of all ranges expressly disclosed herein are expressly disclosed. These are only examples of what is specifically intended and all possible combinations of numerical values between the lowest value and the highest value enumerated are to be considered to be expressly stated in this application in a similar manner.

As used herein, the terms “administration” or “administering” are defined to include an act of providing a compound or pharmaceutical composition of the disclosure to a subject in need of treatment. The phrases “parenteral administration” and “administered parenterally” as used herein means modes of administration other than enteral and topical administration, usually orally or by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticulare, subcapsular, subarachnoid, intraspinal and infrasternal injection and infusion. The phrases “systemic administration,” “administered systemically,” “peripheral administration” and “administered peripherally” as used herein mean the administration of a compound, drug or other material other than directly into the central nervous system, such that it enters the subject's system and, thus, is subject to metabolism and other like processes, for example, subcutaneous administration.

As used herein, the term “comprising,” which is used interchangeably with “including,” “containing,” or “characterized by,” is inclusive or open-ended language and does not exclude additional, unrecited elements or method steps.

As used herein, the term “consisting of” excludes any element, step, or ingredient not specified in the claim.

As used herein, the phrase “consisting essentially of” limits the scope of a claim to the specified materials or steps and those that do not materially affect the basic and novel characteristics of the claimed disclosure. The present disclosure contemplates embodiments of the disclosure compositions and methods corresponding to the scope of each of these phrases. Thus, a composition or method comprising recited elements or steps contemplates particular embodiments in which the composition or method consists essentially of or consists of those elements or steps.

As used herein, the term “chronic kidney disease” or “CKD” refers to the progressive loss of kidney function over time. In some embodiments, CKD may include but is not limited to hyperphosphatemia (i.e., for example, >4.6 mg/dl) or low glomerular filtration rates (i.e., for example, <90 ml/minute per 1.73 m2 of body surface). However, many CKD patients may have normal serum phosphate levels in conjunction with a sustained reduction in glomerular filtration rate for 3 or more months, or a normal glomerular filtration rate (GFR) in conjunction with sustained evidence of a structural abnormality of the kidney. Common symptoms of chronic kidney disease include tiredness, nausea, urine-like odor to the breath, bone pain, abnormally dark or light skin, itching, restless leg syndrome, blood in stools, bruising easily, pedal edema, and peripheral edema.

As used herein, the term “subject,” refers to any individual or patient to which the subject methods are performed. Generally the subject is human, although as will be appreciated by those in the art, the subject may be an animal. Thus other animals, including mammals such as rodents (including mice, rats, hamsters and guinea pigs), cats, dogs, rabbits, farm animals including cows, horses, goats, sheep, pigs, etc., and primates (including monkeys, chimpanzees, orangutans and gorillas) are included within the definition of subject.

As used herein, the term “therapeutically effective amount” or “effective amount” means the amount of a compound or pharmaceutical composition that will elicit the biological or medical response of a tissue, system, animal or human that is being sought by the researcher, veterinarian, medical doctor or other clinician. Thus, the term “therapeutically effective amount” is used herein to denote any amount of a formulation that causes a substantial improvement in a disease condition when applied to the affected areas repeatedly over a period of time. The amount will vary with the condition being treated, the stage of advancement of the condition, and the type and concentration of formulation applied. Appropriate amounts in any given instance will be readily apparent to those skilled in the art or capable of determination by routine experimentation.

As used herein, the term “therapeutic effect,” encompasses a therapeutic benefit and/or a prophylactic benefit as described herein.

As used herein, the terms “reduce” and “inhibit” are used together because it is recognized that, in some cases, a decrease can be reduced below the level of detection of a particular assay. As such, it may not always be clear whether the expression level or activity is “reduced” below a level of detection of an assay, or is completely “inhibited.” Nevertheless, it will be clearly determinable, following a treatment according to the present methods.

As used herein, the term “treatment” or “treating” means to administer a composition or drug to a subject or a system with an undesired condition. The condition can include a disease or disorder. “Prevention” or “preventing” means to administer a composition to a subject or a system at risk for the condition. The condition can include a predisposition to a disease or disorder. The effect of the administration of the composition to the subject (either treating and/or preventing) can be, but is not limited to, the cessation of one or more symptoms of the condition, a reduction or prevention of one or more symptoms of the condition, a reduction in the severity of the condition, the complete ablation of the condition, a stabilization or delay of the development or progression of a particular event or characteristic, or minimization of the chances that a particular event or characteristic will occur.

As used herein, the term “polypeptide” encompasses amino acid chains of any length, including full length sequences in which amino acid residues are linked by covalent peptide bonds. Polypeptides useful in the present disclosure may be purified natural products, or may be produced partially or wholly using recombinant or synthetic techniques. The term may refer to a polypeptide, an aggregate of a polypeptide such as a dimer or other multimer, a fusion polypeptide, a polypeptide fragment, a polypeptide variant, or derivative thereof. Reference to other polypeptides of the disclosure or other polypeptides described herein should be similarly understood.

As used herein, the term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, α-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the disclosure.

As used herein, the term “antibody” refers to polyclonal and monoclonal antibodies and fragments thereof, and immunologic binding equivalents thereof. In some embodiments, “antibody” refers to a homogeneous molecular entity, or a mixture such as a polyclonal serum product made up of a plurality of different molecular entities, and broadly encompasses naturally-occurring forms of antibodies (for example, IgG, IgA, IgM, IgE) and recombinant antibodies such as single-chain antibodies, chimeric and humanized antibodies and multi-specific antibodies. In some embodiments, the term “antibody” also refers to fragments and derivatives of all of the foregoing, and may further comprise any modified or derivatised variants thereof that retains the ability to specifically bind an epitope. Antibody derivatives may comprise a protein or chemical moiety conjugated to an antibody. A monoclonal antibody is capable of selectively binding to a target antigen or epitope. Antibodies may include, but are not limited to polyclonal antibodies, monoclonal antibodies (mAbs), humanized or chimeric antibodies, camelid antibodies, single chain antibodies (scFvs), Fab fragments, F(ab′)2 fragments, disulfide-linked Fvs (sdFv) fragments, for example, as produced by a Fab expression library, anti-idiotypic (anti-Id) antibodies, intrabodies, nanobodies, synthetic antibodies, and epitope-binding fragments of any of the above.

As used herein, the term “ELISA” means an enzyme linked immunosorbent assay, a type of competitive binding assay comprising antibodies and a detectable label used to quantitate the amount of an analyte in a sample.

As used herein, the term “capture antibody” as used herein means an antibody which is typically immobilized on a solid support such as a plate, bead or tube, and which antibody binds to and captures analyte(s) of interest, for example urine tubule bound markers associated with kidney function.

As used herein, the term “detection antibody” means an antibody comprising a detectable label that binds to analyte(s) of interest. The label may be detected using routine detection means for a quantitative, semi-quantitative or qualitative measure of the analyte(s) of interest, for example urine tubule markers associated with kidney function.

As used herein, the term “marker” or “biomarker” in the context of an analyte means any antigen, molecule or other chemical or biological entity that is specifically found in circulation or associated with a particular tissue (e.g., urinary tubules) that it is desired to be identified in a biological sample or on a particular tissue affected by a disease or disorder, for example CKD.

As used herein, the terms “manage”, “managing”, and “management” in the context of the administration of a therapy to a subject refer to the beneficial effects that a subject derives from a therapy (e.g., a prophylactic or therapeutic agent) or a combination of therapies, while not resulting in a cure of the disease or condition. In various examples, a subject is administered one or more therapies (e.g., one or more prophylactic or therapeutic agents) to “manage” the disease or condition so as to prevent the progression or worsening of the disease or condition.

As used herein, the terms “sample” and “biological sample” refer to any sample suitable for the methods provided by the present disclosure. In various embodiments, the biological sample of the present disclosure is a sample of bodily fluid, e.g., serum, plasma, sputum, lung aspirate, urine, and ejaculate.

As used herein, the term “normal samples” or “corresponding normal samples” means biological samples of the same type as the biological sample obtained from the subject. In some embodiments, the corresponding normal sample is a sample obtained from a healthy individual. Such corresponding normal samples can, but need not be, from an individual that is age-matched and/or of the same sex as the individual providing the sample being examined.

As used herein, the term “intensive BP arm” means eligible participants who were randomly assigned to a systolic blood-pressure target of 120 mmHg or less prior to receiving the antihypertensive regimens.

As used herein, the term “standard BP arm” means eligible participants who were randomly assigned to a systolic blood-pressure target of 140 mmHg or less prior to receiving the antihypertensive regimens.

As used herein, the term “hemodynamic perturbation” or “hemodynamic change” refers to changes in the baseline levels of renal vascular resistance, renal plasma blood flow, glomerular filtration rate, sodium excretion, and filtration fraction.

As used herein, the term “antihypertensive regimen” refers to one or a combination of antihypertension medications, including, but not limited to diuretics (thiazide-type diuretics, loop diuretics, and beta-adrenergic blockers), renin-angiotensin system (RAS) antagonists, β-adrenergic blockers, α-adrenergic blockers and calcium channel blockers. In some embodiments, the antihypertensive regimens includes chlorthalidone, amlodipine, and azilsartan.

As used herein, the term “Systolic Blood Pressure Intervention Trial (SPRINT)” refers to a clinical trial for hypertension treatment research that reevaluated the target systolic blood pressure (SBP) used in clinics for treating hypertensive patient.¹³ The standard target SBP for physicians treating patients was 140 mmHg, but SPRINT showed that targeting a patient's SBP down to less than 120 mmHg provided numerous health risk improvements. SPRINT used 9361 hypertensive patients above the age of 50 and showed that a more aggressive targeted SBP of 120 mmHg reduced the cardiovascular risk by 25-30%. SPRINT concluded that a more intensive management of hypertension that lowered target number for SBP to about 120 mmHg or less, significantly reduced rates of major cardiovascular events, and lowers the risk of death from any cause in a group of adults aged 50 years and older when compared to the standard management of about 140 mmHg or less.

Recently, SPRINT (Systolic Blood Pressure Intervention Trial) compared the effects of intensive BP lowering (systolic BP (SBP) target of <120 mm Hg) to standard BP control (SBP <140 mm Hg) on risk for CVD events.¹³ SPRINT enrolled hypertensive individuals without diabetes or prior stroke, but with high CVD risk. Approximately 30% (n=2,646) had CKD at baseline. SPRINT was terminated early at the recommendation of the data safety monitoring board due to substantial benefit for the primary CVD end point and lower mortality risk in patients randomly assigned to the intensive BP arm. Comparing subgroups with and without CKD at baseline, there was no evidence of heterogeneity for the CVD end point, and yet the intensive arm experienced more rapid loss of estimated glomerular filtration rate (eGFR) and higher risk for acute kidney injury (AM).^{13,14} The effect of intensive BP lowering on eGFR was most pronounced during the first six months of treatment, which has led to speculation that the change may represent hemodynamic effects of more intensive BP lowering on eGFR rather than intrinsic kidney damage.¹⁴

SPRINT and several other trials evaluating more versus less intensive BP lowering have demonstrated that intensive BP lowering results in acute losses of eGFR.^{8,11,12,14} These eGFR differences appear to persist during follow-up, but with relatively similar slopes across treatment arms after the acute phase.^{11,14} Determining whether intensive BP lowering reflects hemodynamic changes versus intrinsic kidney damage is of high importance given proven benefits in CVD and mortality risk reduction, but perceived potential harm on the kidney with intensive BP lowering.

As used herein, the terms “chronic kidney disease,” “CKD,” or “chronic renal disease,” refer to a progressive loss of kidney function over a period of months or years. In some embodiments, the CKD may be any stage, including, for example, Stage 1, Stage 2, Stage 3, Stage 4, or Stage 5 (also known as established CKD, end-stage renal disease (ESRD), chronic kidney failure (CKF), or chronic renal failure (CRF)). In some embodiment, the CKD may be caused by hypertension treatment and additionally, any one of a number of factors, including, but not limited to, acute kidney injury, causes of acute kidney injury, Type 1 and Type 2 diabetes mellitus leading to diabetic nephropathy, high blood pressure (hypertension), glomerulonephritis (inflammation and damage of the filtration system of the kidneys), polycystic kidney disease, use (e.g., regular and over long durations of time) of analgesics (e.g., acetaminophen, ibuprofen) leading to analgesic nephropathy, atherosclerosis leading to ischemic nephropathy, obstruction of the flow of urine by stones, an enlarged prostate strictures (narrowings), HIV infection, sickle cell disease, illicit drug (e.g., heroin, cocaine) abuse, amyloidosis, kidney stones, chronic kidney infections, and certain cancers.

As used herein, the term “renal impairment” refers to the kidney's inability to perform its job due to the presence of a toxic substance in the blood or an adverse health condition. As used herein, the term “renal failure” means a progressive renal disease, where a patient has experienced a serious renal injury, and the patient's renal functions are at about 50 percent or less.

As defined herein, the term “glomerular filtration rate (GFR)” means the volume of fluid filtered from the renal (kidney) glomerular capillaries into the Bowman's capsule per unit time. In some embodiments, GFR is indicative of overall kidney function. In some embodiments, GFR is calculated by measuring any chemical that has a steady level in the blood, and is freely filtered but neither reabsorbed nor secreted by the kidneys. In some embodiments, GFR measures the rate at which the chemical reaches the urine, and the quantity of the substance in the urine that originated from a calculable volume of blood. The GFR is typically recorded in units of volume per time, e.g., milliliters per minute. The formula below can be used: GFR=(Urine ConcentrationxUrine Volume)/Plasma Concentration.

In some embodiments, the GFR is determined by injecting inulin into the plasma. Since inulin is neither reabsorbed nor secreted by the kidney after glomerular filtration, its rate of excretion is directly proportional to the rate of filtration of water and solutes across the glomerular filter. In a healthy subject, the GFR is between 90-125 mL/min/1.73 m². In some embodiments, the GFR of a normal subject is 100-125 mL/min/1.73 m2. In some embodiments, GFR measurement involves isotopic such as chromium-51 (⁵¹Cr)-EDTA, iodine-125 (¹²⁵I)-iothalamate or technetium-99m diethylenetriaminepentaacetic acid ([^{99m}Tc]DTPA). In some embodiments, GFR measurement involves non-isotopic such as iohexol or iothalamate.

In some embodiments, the “estimated glomerular filtration rate (eGFR)” is calculated by screening serum creatinine values based on e.g., the Chronic Kidney Disease Epidemiology Collaboration (CKD-EPI) equation, the Cockcroft-Gault formula or the Modification of Diet in Renal Disease (MDRD) formula, which are all known in the art.

Unless defined otherwise, all technical and scientific terms used herein have the same meaning as commonly understood by one of ordinary skill in the art to which this disclosure belongs. Although any methods and materials similar or equivalent to those described herein can be used in the practice or testing of the disclosure, the preferred methods and materials are now described.

### Serum Creatinine

For over 70 years, prior to the present disclosure, serum creatinine remained the primary index for detection and monitoring of kidney disease. Tubulo-interstitial damage and fibrosis were highly prognostic for subsequent kidney failure in kidney biopsy studies, yet this pathology was invisible to the clinician in the absence of a biopsy. The limitations of serum creatinine as the cornerstone of clinical diagnosis and monitoring of kidney disease have been widely described. Beyond the consequences of its influence by muscle mass, diet, and tubule secretion, serum creatinine changes cannot differentiate between benign hemodynamic changes versus intrinsic kidney injury. In oncology and cardiology, clinical care was transformed by embracing and utilizing biomarkers that provide new pathological insights. Nephrology has been slow to adopt and integrate new markers for clinical care. Within nephrology, each new biomarker is typically held to the standard of whether it, as an individual marker, can improve clinical decision making. However, single biomarkers rarely are able to achieve that standard of risk discrimination or diagnostic utility.

The present disclosure provides a panel of biomarkers that reflect distinct aspects of kidney tubule function and injury. As disclosed herein, these novel markers would provide additional information on risk of CKD progression and associated adverse clinical endpoints, above and beyond eGFR and albuminuria. These kidney tubule biomarkers also provide new opportunities to monitor response to therapeutics used to treat CKD patients. Accordingly, the present disclosure provides a broader assessment of kidney health that moves beyond a focus on the glomerulus, and highlights how such biomarkers would improve diagnostic accuracy and earlier assessment of therapeutic efficacy or harm in CKD patients.

The kidney has many biological functions carried out by tubule cells, and the vast majority of energy expenditure within the kidney is devoted to the processes of electrolyte transport, acid-base homeostasis, and endocrine functions in kidney tubules. Damage to the kidney is also not limited to the glomerulus; rather, on kidney biopsy, tubular atrophy and tubule-interstitial fibrosis are common findings in virtually all forms of CKD,³²⁻³⁴ and their severity have consistently proven to be the most reliable features for prediction of progression to end stage kidney disease (ESKD) in nearly all etiologies of kidney disease evaluated^{33,35-36}. These important components of kidney damage are not fully captured by either lower eGFR or greater ACR. For example, a large study of 1203 biopsies from healthy kidney donor candidates found that tubule-interstitial fibrosis was present in 28% overall, ranging from 3% in the 20-29 age group to 73% in those aged 70-79 years. However, the severity of tubular atrophy and fibrosis on biopsy had no association with measured (iothalamate) GFR³⁴. Therefore, despite their prognostic importance in nearly all forms of CKD, tubular disease cannot be reliably detected by the standard clinical measures of glomerular health (eGFR and ACR) and is invisible to the clinician except in the rare instances when a biopsy is obtained.

Accordingly, the present inventors began exploring individual biomarkers that could give insights to unique aspects of kidney tubule health. Similar to the clinically available biomarkers of glomerular health (eGFR and ACR), these newer biomarkers could be characterized into two broad groups reflecting aspects of tubule injury and tubule dysfunction. When valuating individual biomarkers, signals with clinically important outcomes including CKD progression, CVD, and mortality that were evident even after accounting for eGFR, ACR, and standard CKD risk factors were consistently observed. Thus, a research program to explore a paradigm that maximizes the diagnosis, treatment, and prevention of kidney disease was launched to develop a global assessment of kidney health that extends beyond the glomerulus and assesses the health of kidney tubules (FIG. 2). As described herein, multiple non-invasive measures of tubule dysfunction and injury concurrently were combined with the goal of improving risk assessment for adverse kidney outcomes and related end-points including CVD and heart failure. Provided herein, are novel biomarkers that provide new tools for monitoring CKD therapy.

### Measures of Tubule Injury and Dysfunction

Kidney tubule biomarkers can be generally classified as either reflecting the processes of direct tissue injury and repair in the tubule-interstitium, or as measuring unique functions that are performed by the kidney tubule cells. While some require blood measurements, most can be measured in the urine. Although the kidney damage biomarkers were initially pursued as early indicators of acute kidney injury (AKI) in hospitalized patients, they can also be measured reliably in the ambulatory setting. Collectively, the group of biomarkers disclosed herein quantify the severity of tubule cell injury (e.g. Kidney Injury Marker [KIM] 1), the capacity for the tubules to repair themselves from injury (e.g. epidermal growth factor [EGF]), and the extent of inflammation and fibrotic activity (e.g. monocyte chemoattractant protein [MCP] 1) within the tubulo-interstitial space.

The functions of the kidney tubules are broad, as they are critical to homeostatic control. For example, serum sodium, potassium and calcium concentrations largely reflect unique aspects of the kidney tubules' ability to regulate each of these electrolytes. Additional functions of the kidney tubules are not routinely measured clinically, but are available currently as research assays. These include assessment of the proximal tubules' capacity to reabsorb filtered small molecular weight proteins (e.g. alpha-1 microglobulin); the capacity of the proximal tubule to secrete endogenous metabolites (e.g. Hippurate) or exogenous compounds (e.g. furosemide); the production of proteins required to maintain impermiability of water to distal tubule segments and protect against infection (e.g. uromodulin); and ammonium production as a marker of the kidney tubules' ability to excrete net acids.

Accordingly, the novel tubule biomarkers disclosed herein should give insight to the prognosis of the kidney itself, and tightly aligned end points such as CVD and heart failure. As disclosed herein, the design for evaluating novel CKD prognostic biomarkers was to measure several biomarkers from stored frozen specimens and to compare associations with longitudinal adverse outcomes. For monitoring medication effects, stored specimens were crucial because the biomarker concentrations can be compared before and after treatment initiation in order to distinguish salient or harmful effects to the kidney based on dynamic changes of the candidate measures.

eGFR Loss and Kidney Health

During the past decade, several urinary biomarkers of kidney tubule function and injury have been identified.¹⁵ Although evaluated initially as diagnostic tests for AKI, subsequent studies have demonstrated that higher urine concentrations of these markers also predict more rapid loss of kidney function in community-living individuals without AKI.^{16,17} Because abnormal levels of these biomarkers would suggest intrinsic kidney tubule cell injury and/or dysfunction, they provide an opportunity to assess the influence of intensive BP lowering on kidney health above and beyond eGFR loss.

It was hypothesized, however, that the predominant cause for the greater change in eGFR in the intensive arm of SPRINT reflected hemodynamic changes. Therefore, urinary biomarkers that reflect kidney tubule function, inflammation, injury, and repair were measured in a subset of SPRINT participants with CKD at baseline to identify a panel of specific markers that can be used to differentiate between hemodynamic changes in serum creatinine from intrinsic kidney damage.

As described herein, the effects of random assignment to the intensive SBP-lowering arm of SPRINT on urinary markers of kidney tubule function, injury, and repair in participants with CKD were evaluated. It was found that random assignment to the intensive SBP arm was associated with a decline in eGFR by year 1 that persisted over 4 years. It was concurrently found that concentrations of two kidney tubule function markers, urinary B2M and A1M, were lower in the intensive arm at 1 year, an effect that was attenuated and no longer evident by 4 years after randomization. None of the kidney tubule cell biomarkers had a statistically higher concentration in the intensive arm at either the year 1 or year 4 follow-up visits despite the loss of eGFR in the intensive arm.

As demonstrated herein, eight urinary markers of intrinsic kidney tubule damage were evaluated. Despite declines in eGFR in the intensive arm, no evidence was found that levels of any of the eight kidney tubule biomarkers were elevated compared to the standard SBP arm, after either 1 or 4 years of intensive BP lowering. Because higher urine levels of these kidney tubule markers have been linked to CKD progression, dialysis therapy initiation, and adverse health outcomes,^{16,17,24-27} the present results provide reassurance that the eGFR decline with intensive BP lowering is likely predominantly hemodynamic in nature.

Levels of two of the biomarkers (urinary B2M and A1M) were significantly lower, rather than higher, in the intensive BP arm at year 1. These biomarkers of proximal tubule function share similar properties in their renal handling and therefore give insights to the biology responsible for changes in eGFR with intensive BP lowering. Both B2M and A1M are serum proteins that are filtered by the glomerulus and then reabsorbed by the proximal tubule. In contrast, the other six urinary tubule biomarkers are produced in kidney tissue in response to damage, inflammation, and repair and are not known to be filtered at the glomerulus. Accordingly, intensive SBP lowering results in a hemodynamic decrease in GFR, which not only lowers creatinine filtration, but also lowers B2M and A1M filtration in the presence of preserved tubular reabsorptive capacities, resulting in lower urine concentrations. These findings were reinforced by the analyses stratified by the magnitude of change in eGFR and SBP. Participants in the intensive arm who experienced the largest reductions in eGFR and SBP during the trial also experienced the greatest reductions in urinary B2M and A1M levels. Similarly, the finding of lower albuminuria in the intensive SBP arm may be as a consequence of decrease in glomerular capillary pressure or inhibition of podocyte damage and myofibroblast transformation.¹⁴ Accordingly, the novel biomarkers disclosed herein have clinical implications because they will provide reassurance to clinicians and patients when they consider continuation of intensive BP-lowering therapy even if eGFR increases within the range observed within SPRINT. Furthermore, they are good alternative to serum creatinine measurement.

It was further hypothesized that chronic hemodynamic perturbations would not lead to tubular damage. For example, recent studies evaluating sodium/glucose co-transporter 2 (SGLT2) inhibitors show acute hemodynamic effects on eGFR that persist for years, but then rapidly resolve after drug discontinuation.²⁸⁻³⁰ SGLT2 inhibitors are associated with lower risk for end-stage kidney disease.²⁹ Therefore hemodynamic effects on eGFR may persist for years without necessarily causing tubule damage. In some embodiments, the tubule health markers disclosed herein will have utility in the assessment of intrinsic versus hemodynamic changes in kidney function in other settings that are known to influence renal perfusion. In some embodiments, the tubule health markers disclosed herein will be used in monitoring of patients initiated on treatment with angiotensin-converting enzyme inhibitors, nonsteroidal anti-inflammatory medications, SGLT2 inhibitors, and other drugs.

The present disclosure has several strengths. First, multiple urinary kidney tubule markers that reflect unique aspects of kidney tubule biology, including tubule function, injury, inflammation, and repair were longitudinally assessed. Second, kidney tubule cell damage, atrophy, and tubulointerstitial fibrosis are hallmarks of nearly all forms of progressive CKD, and the urinary biomarkers evaluated here are known to be associated with CKD progression above and beyond eGFR and urinary albumin-creatinine ratio (ACR). Third, the randomized trial design, 4 years of follow-up, and consistent directions of the observed associations across the panel of biomarkers are additional strengths. The kidney tubule marker measurements were performed en bloc to minimize the influence of laboratory drift and more closely reflect biological changes. Biomarkers were measured twice in each sample and results were averaged to improve precision. The randomized trial design minimizes the influence of bias or unmeasured confounding the results provided herein.

Although intensive SBP lowering resulted in reductions in eGFR, evidence that SBP lowering induced kidney tubule cell damage was not found based on evaluation of the 8 distinct kidney tubule biomarkers disclosed herein. Intensive BP lowering was associated with lower concentrations of 2 urinary biomarkers that are filtered at the glomerulus and reabsorbed at the proximal tubule. Accordingly, reductions in eGFR observed with intensive BP lowering reflect hemodynamic changes rather than intrinsic kidney cell damage in persons with CKD. Thus, the 8 urinary biomarkers of the present disclosure reflect different aspects of kidney tubule function and damage.

### Modes For Carrying Out the Disclosure

### Method of Treating Diabetes Mellitus

The present disclosure provides a method for treating diabetes mellitus in a subject in need thereof comprising, or alternatively consisting essentially of, or yet further consisting of: (a) administering a therapeutic regimen to the subject after the levels of one or more of: α₁-microglobulin (A1M), β₂-microglobulin (B2M), kidney injury molecule 1 (KIM-1), interleukin 18 (IL-18), monocyte chemoattractant protein 1 (MCP-1), neutrophil gelatinase-associated lipocalin (NGAL), uromodulin (UMOD), and human cartilage glycoprotein 39 (YKL-40) were measured in a first biological sample isolated from the subject; and (b) comparing the levels of A1M, B2M, KIM-1, IL-18, MCP-1, NGAL, UMOD, and YKL-40 in a second biological sample from the subject to the levels of the first biological sample.

In some embodiments, α1-microglobulin (A1M), β2-microglobulin (B2M), kidney injury molecule 1 (KIM-1), interleukin 18 (IL-18), monocyte chemoattractant protein 1 (MCP-1), neutrophil gelatinase-associated lipocalin (NGAL), uromodulin (UMOD), and/or human cartilage glycoprotein 39 (YKL-40) are distinct urinary markers that reflect aspects of kidney tubule biology, including tubule function, injury, inflammation, and repair. In some embodiments, the urinary marker is B2M or MM. B2M and A1M are low-molecular-weight proteins that are freely filtered at the glomerulus and then reabsorbed by the proximal tubule. In some embodiments, higher levels of B2M and/or A1M in urine are associated with kidney function decline.¹⁹ In some embodiments, higher urine A1M correlates with worse proximal tubule reabsorptive function. In some embodiments, higher urine A1M is associated with future development of AKI. In some embodiments, higher urine A1M is associated with higher risk for CVD events.

In some embodiments, the urinary marker is UMOD. UMOD is a 95-kDa glycoprotein synthesized exclusively by renal epithelial cells. Higher UMOD levels are associated with kidney size and eGFR, and lower UMOD levels are independently associated with CKD progression.²° UMOD is required to maintain water impermeability in the distal tubule segments and protect kidneys against infection. In particular, UMOD regulates ion transport in the thick ascending limb, immunomodulation, and protection against urinary tract infections and kidney stones. In some embodiments, higher urine concentrations of UMOD are strongly associated with slower decline in eGFR. In some embodiments, higher UMOD concentrations are strongly associated with higher risk for CVD events. In some embodiments, lower urinary UMOD correlates with reduced tubule synthetic function. In some embodiments, lower urinary UMOD is associated with future development of AKI.

In some embodiments, the urinary marker is IL-18, KIM-1, or NGAL. NGAL, KIM-1, IL-18, MCP-1, and YKL-40 are produced within the kidney tissue in response to injury, inflammation, or repair. In some embodiments, NGAL, KIM-1, IL-18, MCP-1, or YKL-40 is a marker of proximal tubule injury. In some embodiments, urine levels of IL-18, KIM-1, and NGAL increase by several-fold in response to ischemic or inflammatory kidney injury.^{21,22} In some embodiments, higher levels of KIM-1 or IL-18 are associated with elevated risk for CKD progression. In some embodiments, the marker is Interleukin-18 (IL-18), a member of the IL-1 family of cytokines. IL-18 is synthesized as an inactive 23-kDa precursor by several tissues including monocytes, macrophages, and proximal tubular epithelial cells, and is processed into an active 18.3 kDa cytokine by caspase-1. IL-18 functions as a mediator of renal ischemia—reperfusion injury, inducing acute tubular necrosis, and neutrophil and monocyte infiltration of the renal parenchyma. In some embodiments, the marker is NGAL. NGAL is a member of the lipocalin family of proteins. NGAL a 25 kDa protein produced by injured nephron epithelia. NGAL is normally produced and secreted by renal cells at low levels, but the amount produced and secreted into the urine and serum increases dramatically after ischemic, septic, or nephrotoxic injury of the kidneys. In some embodiments, the marker is KIM-1. KIM-1 is a type 1 transmembrane protein, with an immunoglobulin and mucin domain that is expressed on the surface of renal epithelial cells. KIM-1 expression level is undetectable in healthy renal cells, but its expression is upregulated within hours following kidney injury. Renal injury also causes, KIM-1 immunoglobulin and mucin domains to shed from the cells and enter urine. In some embodiment, the marker is YKL-40. YKL-40 is a chitinase 3-like-protein that plays an important role in acute kidney injury (AKI) and repair. YKL-40 is produced by activated macrophages and neutrophils and is expressed in a wide range of inflammatory conditions , such as AKI.

In some embodiments, the urinary marker is MCP-1. MCP-1 is a chemokine that attracts macrophages to the site of injury, and its levels strongly correlate with CKD progression in kidney transplant recipients.²³ In some embodiments, the urinary marker is YKL-40. YKL-40 functions as a mediator of the reparative response to tubular injury.²⁴ In some embodiments, the selected urinary markers, A1M, B2M, KIM-1, IL-18, MCP-1, NGAL, UMOD, or YKL-40 measures the interlinked axes of inflammation, tubular injury and atrophy, and tubulointerstitial fibrosis, which are hallmarks of progressive CKD.

In some embodiments, the method includes measuring the levels of one or more of α₁-microglobulin (A1M), β₂-microglobulin (B2M), kidney injury molecule 1 (KIM-1), interleukin 18 (IL-18), monocyte chemoattractant protein 1 (MCP-1), neutrophil gelatinase-associated lipocalin (NGAL), uromodulin (UMOD), and human cartilage glycoprotein 39 (YKL-40) in a first biological sample from the subject; and comparing the measured levels against those of a second biological sample from the subject. In some embodiment, a single marker is used. In some embodiments, a panel comprising at least two markers, at least three, at least four, at least five, at least six, at least seven or at least eight biomarkers are used. In some embodiments, the panel of biomarkers comprises at least one marker of proximal tubule injury and at least one marker of kidney tubule function. In some embodiments, the panel of biomarkers is combined with eGFR measurement and/or albumin-to-creatinine ratio. In some embodiments, the levels of one or more comprises the levels of KIM-1, IL-18, MCP-1, NGAL, UMOD, and YKL-40. In some embodiments, the levels of one or more comprises the levels of a combination of KIM-1, NGAL and UMOD; MCP-1, IL-18 and YKL-40; UMOD, MCP-1, and IL-18; NGAL, MCP-1, and IL-18; or KIM-1, MCP-1 and IL-18. In some embodiments, the measured levels of one or more comprises: (1) the measured levels of at least one of UMOD, NGAL, KIM-1, and (2) the measured levels of at least one of MCP-1, IL-18 or YKL-40.

In some embodiments, the panel of biomarkers further comprises cystatin C, clusterin, osteopontin, EGF, trefoil factor 3, or chitinase 3-like protein 1. In some embodiments, the one or more markers are A1M, B2M, KIM-1, IL-18, MCP-1, NGAL, UMOD, YKL-40, eGFR, albumin, creatinine, albumin-creatinine ratio (ACR), cystatin C, clusterin, osteopontin, EGF, trefoil factor 3, and chitinase 3-like protein 1. In some embodiment, the one or more markers are a combination of A1M, B2M, KIM-1, IL-18, MCP-1, NGAL, UMOD, YKL-40 eGFR, or albumin-creatinine ratio (ACR).

### Detection of Biomarkers

In some embodiments, a biological sample is first obtained from a subject suspected of having a disease or condition described herein. In some embodiments, biological samples contemplated by the present disclosure comprise, consists essentially of, or yet further consist of, but are not limited to, cell sample, tissue sample, tumor biopsy, liquid samples such as blood and other liquid samples of biological origin (including, but not limited to, peripheral blood, sera, plasma, ascites, urine, cerebrospinal fluid (CSF), sputum, saliva, bone marrow, synovial fluid, aqueous humor, amniotic fluid cerumen, breast milk, broncheoalveolar lavage fluid, semen, prostatic fluid, cowper's fluid or pre-ejaculatory fluid, female ejaculate, sweat, tears, cyst fluid, pleural and peritoneal fluid, pericardial fluid, ascites, lymph, chyme, chyle, bile, interstitial fluid, menses, pus, sebum, vomit, vaginal secretions/flushing, synovial fluid, mucosal secretion, stool water, pancreatic juice, lavage fluids from sinus cavities, bronchopulmonary aspirates, blastocyl cavity fluid, or umbilical cord blood.

In some embodiments, the sample is a tumor biopsy. In some embodiments, the sample is a liquid sample. In some cases, the sample is a cell-free DNA sample. In some embodiments, the sample is urine. In some embodiments, the sample is blood. In some embodiments, the sample is plasma. Plasma biomarkers of kidney tubule injury are well-characterized) and found that higher levels of plasma KIM-1 strongly correlated with CKD progression.¹² In some embodiments, plasma concentrations of biomarkers specific to the kidney tubules have stronger associations than urine concentrations.

Methods of detecting analyte levels in biological samples are well known to a skilled artisan. Immunoassay devices and methods can be used. See, e.g., U.S. Pat. Nos. 6,143,576; 6,113,855; 6,019,944; 5,985,579; 5,947,124; 5,939,272; 5,922,615; 5,885,527; 5,851,776; 5,824,799; 5,679,526; 5,525,524; and 5,480,792, each of which is hereby incorporated by reference in its entirety. These devices and methods can utilize labeled molecules in various sandwich, competitive, or non-competitive assay formats, to generate a signal that is related to the presence or amount of an analyte of interest. In some embodiments, biosensors and optical immunoassays, may be employed to determine the presence or amount of analytes without the need for a labeled molecule. See, e.g., U.S. Pat. Nos. 5,631,171; and 5,955,377, each of which is hereby incorporated by reference in its entirety, including all tables, figures and claims. One skilled in the art also recognizes that robotic instrumentation including but not limited to Beckman ACCESS®, Abbott AXSYM®, Roche ELECSYS®, Dade Behring STRATUS® systems are among the immunoassay analyzers that are capable of performing the immunoassays taught herein.

In some embodiment, methods for detecting an analyte in a biological sample comprises or consists essentially of enzyme-linked immunosorbent assay (ELISA), radioimmunoassays (RIAs), competitive binding assays, western blot, immunoprecipitation, and immunofluorescence using detection reagents such as an antibody or protein binding agents. In some embodiments, the biomarkers are analyzed using an immunoassay. The presence or amount of a marker is generally determined using antibodies specific for each marker and detecting specific binding. Specific immunological binding of the antibody to the marker can be detected directly or indirectly. Direct labels include fluorescent or luminescent tags, metals, dyes, radionuclides, and the like, attached to the antibody. Indirect labels include various enzymes well known in the art, such as alkaline phosphatase, horseradish peroxidase. Alternatively, a peptide can be detected in a biological sample from a subject by introducing into the sample a labeled anti-peptide antibody and other types of detection agent. For example, the antibody can be labeled with a detectable marker whose presence in the sample is detected by standard imaging techniques.

In some embodiments, the biomarkers are analyzed by measuring the marker RNA levels. Expression levels or abundance of the one or more makers can be determined by direct measurement of expression at the protein or mRNA level, for example by microarray analysis, quantitative PCR analysis, or RNA sequencing analysis. Alternatively, labeled antibody systems may be used to quantify target protein abundance in the cells, followed by immunofluorescence analysis, such as FISH analysis.

In some embodiments, the first biological sample is taken prior to administering a therapeutic regiment. In some embodiments, the second biological sample is taken after administering the therapeutic regiment. In some embodiments, a lack of elevated levels of KIM-1, IL-18, MCP-1, NGAL, UMOD, and/or YKL-40 in the second biological sample as compared to the first biological sample is indicative of continued kidney health and the therapeutic regimen should be continued. In various embodiments, an increase in the measured levels in the second biological sample as compared to the first biological sample is indicative of progression to chronic kidney disease (CKD) in the subject. In some embodiments, an increase in the measured levels in the second biological sample as compared to the first biological sample is indicative that the treatment for diabetes mellitus should be discontinued and/or the subject should be administered an alternative therapeutic regimen.

In some embodiments, the method of treatment further comprises or alternatively consists essentially of, or yet further consists of administering the therapeutic regimen to the subject if the levels of KIM-1, IL-18, MCP-1, NGAL, UMOD, and/or YKL-40 in the second biological sample are not elevated when compared to the first biological sample. In some embodiments, the therapeutic regimen causes a decrease in eGFR in the second biological sample when compared to the first biological sample. In some embodiments, the therapeutic regimen causes a decrease in the levels of A1M or B2M in the second biological sample when compared to the first biological sample.

In some embodiments, a subject having been diagnosed with diabetes mellitus will provide a urine sample prior to initiating a therapeutic regimen, and again after being on a therapeutic regimen for a predetermined amount of time. Various methods known in the art can also be utilized to determine the presence of a disease or condition described herein or to determine whether an immune response has been induced in a subject. Assessment of one or more biomarkers associated with a disease or condition, or for characterizing whether an immune response has been induced, can be performed by any appropriate method.

Exemplary predetermined amounts of time useful in the method so of the present disclosure include, but are not limited to, 1 month, 3 months, 6 months, 9 months, 1 year, 2 years, and 3 years, depending on the overall health of the subject. In some embodiments, the predetermined time is selected from 1 month, 3 months, 6 months, 9 months, 1 year, 2 years, or 3 years. In some embodiments, the method of treatment further comprises or alternatively consists essentially of, or yet further consists of administering the therapeutic regimen to the subject if the levels of KIM-1, IL-18, MCP-1, NGAL, UMOD, and/or YKL-40 in the second, third, fourth, sixth or seventh biological sample are not elevated when compared to the last biological sample tested. In some embodiments, the levels of KIM-1, IL-18, MCP-1, NGAL, UMOD, and/or YKL-40 in the biological sample of the subject is measured routinely during treatment. In some embodiments, the levels of KIM-1, IL-18, MCP-1, NGAL, UMOD, and/or YKL-40 in the biological sample of the subject is measured and compared to the last biological sample from the same subject at 1 month, 3 months, 6 months, 9 months, 1 year, 2 years, 3 years, or 4 years interval depending on the overall health of the subject.

### Therapeutic Regimen

In some embodiments, the therapeutic regimen comprises, consists essentially of, or further yet consists of administering a therapeutic selected from sodium glucose transporter 2 (SGLT2) inhibitors, angiotensin-converting enzymes inhibitors, nonsteroidal anti-inflammatory medications, antihypertensive medications, intensive blood pressure lowering medications or a combination thereof.

In some embodiments, the therapeutic regimen comprises administering a therapeutic selected from sodium glucose transporter 2 (SGLT2) inhibitors, metformin, sulphonylureas, nateglinide, repaglinide, thiazolidinediones, pioglitazone PPARα-glucosidase inhibitors, insulin and insulin analogues, Glucagon-like peptide 1 (GLP-1) and GLP-1 analogues, dipeptidyl peptidase-4 (DPP-4) inhibitors or a combination thereof. In some embodiments, the therapeutic regimen comprises administering at least one sodium glucose transporter 2 (SGLT2) inhibitor. In some embodiments, the SGLT2 inhibitor is an FDA-approved therapeutic drug for use along with diet and exercise to lower blood sugar in adults with type 2 diabetes. In some embodiments, the SGLT2 inhibitor lowers blood sugar by causing the kidneys to remove sugar from the body through the urine. In some embodiments, the SGLT2 inhibitor is canagliflozin, dapagliflozin, or empagliflozin. In some embodiments, the SGLT2 inhibitor is a single single-ingredient product. In some embodiments, the SGLT2 inhibitor is in combination with other diabetes medicines such as metformin, sitagliptin, saxagliptin, linagliptin, or alogliptin.

In some embodiments, the therapeutic regimen further comprises, or alternatively consists essentially of, or yet further consists of administering a non- SGLT2 inhibitor therapeutic regimen. In some embodiments, the non-SGLT2 inhibitor therapeutic is for the treatment of diabetes mellitus. In some embodiments, the non-SGLT2 inhibitor therapeutic is selected from metformin, sulphonylureas, nateglinide, repaglinide, thiazolidinediones, pioglitazone PPARa-glucosidase inhibitors, insulin and insulin analogues, Glucagon-like peptide 1 (GLP-1) and GLP-1 analogues or dipeptidyl peptidase-4 (DPP-4) inhibitors. In some embodiments, the treatment regimen is a combination of a SGLT inhibitor and metformin, a SGLT inhibitor and metformin. In some embodiments, the treatment regimen is a combination of a SGLT inhibitor and sulphonylureas, nateglinide, repaglinide, thiazolidinediones, pioglitazone PPARα-glucosidase inhibitors, insulin and insulin analogues, Glucagon-like peptide 1 (GLP-1) and GLP-1 analogues and dipeptidyl peptidase-4 (DPP-4) inhibitors.

In some embodiments, the treatment regimen is a combination of a SGLT inhibitor and a dipeptidyl peptidase-4 (DPP-4) inhibitor. DPP-4 inhibitors are used along with diet and exercise to lower blood sugar in adults with type 2 diabetes. DPP-4 inhibitors are available as single-ingredient products and in combination with other diabetes medicines such metformin, empagliflozin, or pioglitazone. In some embodiments, the treatment regimen is a combination of a SGLT inhibitor and a Glucagon-like peptide 1 (GLP-1) or GLP-2 analogue. In some embodiments, the GPL-1 or GLP-2 analogue is exenatide, exenatide LAR, Lixisenatide, Albiglutide, liraglutide, taspoglutide, or Dulaglutide.

Hypertension is defined as a blood pressure ≥140/90 mmHg, and is an extremely common comorbid condition in diabetes. Hypertension affects about 20-60% of patients with diabetes, depending on obesity, ethnicity, and age. In type 2 diabetes, hypertension is often present as part of the metabolic syndrome of insulin resistance also including central obesity and dyslipidemia. In type 1 diabetes, hypertension may reflect the onset of diabetic nephropathy. Accordingly, in some embodiments of the present disclosure, the therapeutic regimen comprises, consists essentially of, or further yet consist of administering an intensive blood pressure lowering therapy. In some embodiments, the intensive blood pressure lowering therapy is an antihypertensive regimen selected from diuretics, renin-angiotensin system (RAS) antagonists, angiotensin-converting enzyme (ACE) inhibitors, angiotensin receptor blockers (ARBs), β-adrenergic blockers, α-adrenergic blockers, calcium channel blockers, or a combination thereof. In some embodiments, the antihypertensive regimen is selected from thiazide-type diuretics, loop diuretics, chlorthalidone, amlodipine, or azilsartan.

In some embodiments, the antihypertensive regimen is an antihypertensive therapeutic selected from chlorthalidone, chlorothiazide, hydrochlorothiazide, indapamide, metolazone, amiloride hydrochloride, spironolactone, triamterene, furosemide, bumetanide, amiloride hydrochloride, hydrochlorothiazide, spironolactone and hydrochlorothiazide, triamterene and hydrochlorothiazide, acebutolol, atenolol, betaxolol, bisoprolol fumarate, carteolol hydrochloride, metoprolol tartrate, metoprolol succinate, nadolol, penbutolol sulfate, pindolol, propranolol hydrochloride, solotol hydrochloride, or timolol maleate, hydrochlorothiazide and bisoprolol, benazepril hydrochloride, captopril, enalapril maleate, fosinopril sodium, Lisinopril, moexipril, perindopril, quinapril hydrochloride, Ramipril, trandolapril candesartan, eprosartan mesylate, irbesarten, losartan potassium, telmisartan, valsartan, amlodipine besylate, bepridil, diltiazem hydrochloride, felodipine, isradipine, nicardipine, nifedipine, nisoldipine, verapamil hydrochloride, doxazosin mesylate, prazosin hydrochloride, or terazosin hydrochloride, carvedilol, labetalol hydrochloride alpha methyldopa, clonidine hydrochloride, guanabenz acetate, guanfacine hydrochloride, guanadrel, guanethidine monosulfate, reserpine, hydralazine hydrochloride, or minoxidil.

In some embodiments, the subject having diabetes mellitus is at risk of kidney failure when the estimated glomerular filtration rates (eGFR) of the subject is less than 60 ml/min/1.73 m². In some embodiments, eGFR is measured in the first and second biological sample. In some embodiments, eGFR is measured concurrent with A1M, B2M, KIM-1, IL-18, MCP-1, NGAL, UMOD, and/or YKL-40. In some embodiments, treatment with a SGLT inhibitor decreases the eGFR below 60 ml/min/1.73 m². In some embodiments, the eGFR is between 45 ml/min/1.73 m² and 59 ml/min/1.73m². In some embodiments, the eGFR falls below 45 ml/min/1.73m². In some embodiments, the “estimated glomerular filtration rate (eGFR)” is derived at by screening serum creatinine values based on e.g., the Chronic Kidney Disease Epidemiology Collaboration (CKD-EPI) equation, the Cockcroft-Gault formula or the Modification of Diet in Renal Disease (MDRD) formula, which are all known in the art. In some embodiments, the GFR is determined by injecting inulin into the plasma. Since inulin is neither reabsorbed nor secreted by the kidney after glomerular filtration, its rate of excretion is directly proportional to the rate of filtration of water and solutes across the glomerular filter. In some embodiments, GFR measurement involves isotopic such as chromium-51 (⁵¹Cr)-EDTA, iodine-125 (¹²⁵I)-iothalamate or technetium-99m diethylenetriaminepentaacetic acid ([^{99m}Tc]DTPA). In some embodiments, GFR measurement involves non-isotopic such as iohexol or iothalamate. In a healthy subject, the GFR is between 90-125 mL/min/1.73 m². In some embodiments, the GFR of a normal subject is 100-125 mL/min/1.73 m².

In some embodiments, the SGLT inhibitor treatment is maintained if the levels of KIM-1, IL-18, MCP-1, NGAL, UMOD, and/or YKL-40 in subsequent biological samples are stable when compared to the first or previous biological sample and in conjunction with a rapid loss of eGFR. In some embodiments, the SGLT inhibitor treatment is discontinued if the levels of KIM-1, IL-18, MCP-1, NGAL, UMOD, and/or YKL-40 in the second or subsequent biological samples are elevated when compared to the first or previous biological sample in conjunction with a rapid loss of eGFR.

In some embodiments, SGLT inhibitor treatment induced-fall of eGFR is at least 11% reduction, and the levels of KIM-1, IL-18, MCP-1, NGAL, UMOD, and/or YKL-40 in subsequent biological samples are stable when compared to the first or previous biological sample. In some embodiments, the eGFR in the second biological sample is less than 40 ml/min/1.73 m² and the levels of, KIM-1, IL-18, MCP-1, NGAL, UMOD, and/or YKL-40 in subsequent biological samples are stable when compared to the first or previous biological sample. In some embodiments, the method further comprises discontinuing the therapeutic regiment if the levels of KIM-1, IL-18, MCP-1, NGAL, UMOD, and/or YKL-40 in the second biological sample are elevated when compared to the first biological sample and the eGFR is reduced by at least about 11%, or the eGFR in the second biological sample is less than 40 ml/min/1.73 m².

In some embodiments, the subject is at risk of renal impairment or chronic kidney disease (CKD). In some embodiments, the CKD may be any stage, including, for example, Stage 1, Stage 2, Stage 3, Stage 4, or Stage 5 (also known as established CKD, end-stage renal disease (ESRD), chronic kidney failure (CKF), or chronic renal failure (CRF)). In some embodiment, the CKD may be caused by hypertension treatment and additionally, any one of a number of factors, including, but not limited to, acute kidney injury, causes of acute kidney injury, Type 1 and Type 2 diabetes mellitus leading to diabetic nephropathy, high blood pressure (hypertension), glomerulonephritis (inflammation and damage of the filtration system of the kidneys), polycystic kidney disease, use (e.g., regular and over long durations of time) of analgesics (e.g., acetaminophen, ibuprofen) leading to analgesic nephropathy, atherosclerosis leading to ischemic nephropathy, obstruction of the flow of urine by stones, an enlarged prostate strictures (narrowings), HIV infection, sickle cell disease, illicit drug (e.g., heroin, cocaine) abuse, amyloidosis, kidney stones, chronic kidney infections, and certain cancers. In some embodiments, a subject is at risk of renal impairment or chronic kidney disease (CKD), when the eGFR is below 60 ml/min/1.73 m².

### Method of Treating Hypertension

In another aspect, the present disclosure provides a method of treating hypertension in a subject at risk of chronic kidney disease comprising, consisting essentially: (a) administering a therapeutic regimen to the subject after the levels of one or more of: α₁-microglobulin (A1M), β₂-microglobulin (B2M), kidney injury molecule 1 (KIM-1), interleukin 18 (IL-18), monocyte chemoattractant protein 1 (MCP-1), neutrophil gelatinase-associated lipocalin (NGAL), uromodulin (UMOD), and/or human cartilage glycoprotein 39 (YKL-40) were measured in a first biological sample isolated from the subject; and (b) comparing the levels of A1M, B2M, KIM-1, IL-18, MCP-1, NGAL, UMOD, and YKL-40 in a second biological sample from the subject to the levels of the first biological sample.

In some embodiments, the urinary marker is UMOD. UMOD is a 95-kDa glycoprotein synthesized exclusively by kidney tubules. Higher UMOD levels are associated with kidney size and eGFR, and lower UMOD levels are independently associated with CKD progression.²⁰ UMOD is required to maintain water impermeability in the distal tubule segments and protect kidneys against infection. In some embodiments, higher urine concentrations of UMOD are strongly associated with slower decline in eGFR. In some embodiments, lower UMOD concentrations are strongly associated with higher risk for CVD events. In some embodiments, lower urinary UMOD correlates with reduced tubule synthetic function. In some embodiments, lower urinary UMOD is associated with future development of AKI.

In some embodiments, the urinary marker is IL-18, KIM-1, or NGAL. NGAL, KIM-1, IL-18, MCP-1, and YKL-40 are produced within the kidney tissue in response to injury, inflammation, or repair. In some embodiments, NGAL, KIM-1, IL-18, MCP-1, or YKL-40 is a marker of proximal tubule injury. In some embodiments, urine levels of IL-18, KIM-1, and NGAL increase by several-fold in response to ischemic or inflammatory kidney injury.^{21,22} In some embodiments, higher levels of KIM-1 or IL-18 are associated with elevated risk for CKD progression.

In some embodiments, the urinary marker is MCP-1. MCP-1 is a chemokine that attracts macrophages to the site of injury, and its levels strongly correlate with CKD progression in kidney transplant recipients.' In some embodiments, the urinary marker is YKL-40. YKL-40 functions as a mediator of the reparative response to tubular injury.²⁴ In some embodiments, the selected urinary markers, A1M, B2M, KIM-1, IL-18, MCP-1, NGAL, UMOD, or YKL-40 measures the interlinked axes of inflammation, tubular injury and atrophy, and tubulointerstitial fibrosis, which are hallmarks of progressive CKD.

In some embodiments, the method includes measuring the levels of α₁-microglobulin (A1M), β₂-microglobulin (B2M), kidney injury molecule 1 (KIM-1), interleukin 18 (IL-18), monocyte chemoattractant protein 1 (MCP-1), neutrophil gelatinase-associated lipocalin (NGAL), uromodulin (UMOD), and human cartilage glycoprotein 39 (YKL-40) in a first biological sample from the subject; and comparing the measured levels against those of a second biological sample from the subject. In some embodiment, a single marker is used. In some embodiments, a panel comprising at least two markers, at least three, at least four, at least five, at least six, at least seven or at least eight biomarkers are used. In some embodiments, the panel of biomarkers comprises at least one marker of proximal tubule injury and at least one marker of kidney tubule function. In some embodiments, the panel of biomarkers is combined with eGFR measurement and/or albumin-to-creatinine ratio.

In some embodiments, the levels of one or more comprises the levels of KIM-1, IL-18, MCP-1, NGAL, UMOD, and YKL-40. In some embodiments, the levels of one or more comprises the levels of a combination of KIM-1, NGAL and UMOD; MCP-1, IL-18 and YKL-40; UMOD, MCP-1, and IL-18; NGAL, MCP-1, and IL-18; or KIM-1, MCP-1 and IL-18. In some embodiments, the measured levels of one or more comprises: (1) the measured levels of at least one of UMOD, NGAL, KIM-1, and (2) the measured levels of at least one of MCP-1, IL-18 or YKL-40.

In some embodiments, the panel of biomarkers further comprises cystatin C, clusterin, osteopontin, EGF, trefoil factor 3, and chitinase 3-like protein 1. In some embodiments, the one or more markers are A1M, B2M, KIM-1, IL-18, MCP-1, NGAL, UMOD, YKL-40, eGFR, albumin, creatinine, or albumin-creatinine ratio (ACR), cystatin C, clusterin, osteopontin, EGF, trefoil factor 3, and chitinase 3-like protein 1. In some embodiment, the one or more markers are a combination of A1M, B2M, KIM-1, IL-18, MCP-1, NGAL, UMOD, YKL-40 eGFR, or albumin-creatinine ratio (ACR).

### Detection of Biomarkers

In some embodiments, biological samples contemplated by the present disclosure comprise, consists essentially of, or yet further consist of, but are not limited to, cell sample, tissue sample, tumor biopsy, liquid samples such as blood and other liquid samples of biological origin (including, but not limited to, peripheral blood, sera, plasma, ascites, urine, cerebrospinal fluid (CSF), sputum, saliva, bone marrow, synovial fluid, aqueous humor, amniotic fluid cerumen, breast milk, broncheoalveolar lavage fluid, semen, prostatic fluid, cowper's fluid or pre-ejaculatory fluid, female ejaculate, sweat, tears, cyst fluid, pleural and peritoneal fluid, pericardial fluid, ascites, lymph, chyme, chyle, bile, interstitial fluid, menses, pus, sebum, vomit, vaginal secretions/flushing, synovial fluid, mucosal secretion, stool water, pancreatic juice, lavage fluids from sinus cavities, bronchopulmonary aspirates, blastocyl cavity fluid, or umbilical cord blood.

In some embodiments, the sample is a tumor biopsy. In some embodiments, the sample is a liquid sample. In some cases, the sample is a cell-free DNA sample. In some embodiments, the sample is urine. In some embodiments, the sample is blood. In some embodiments, the sample is plasma. Plasma biomarkers of kidney tubule injury are well-characterized.) and found that higher levels of plasma KIM-1 strongly correlated with CKD progression.¹² In some embodiments, plasma concentrations of biomarkers specific to the kidney tubules have stronger associations than urine concentrations.

Methods of detecting analyte levels in biological samples are well known to a skilled artisan. Immunoassay devices and methods can be used. See, e.g., U.S. Pat. Nos. 6,143,576; 6,113,855; 6,019,944; 5,985,579; 5,947,124; 5,939,272; 5,922,615; 5,885,527; 5,851,776; 5,824,799; 5,679,526; 5,525,524; and 5,480,792, each of which is hereby incorporated by reference in its entirety. These devices and methods can utilize labeled molecules in various sandwich, competitive, or non-competitive assay formats, to generate a signal that is related to the presence or amount of an analyte of interest. In some embodiments, biosensors and optical immunoassays, may be employed to determine the presence or amount of analytes without the need for a labeled molecule. See, e.g., U.S. Pat. Nos. 5,631,171; and 5,955,377, each of which is hereby incorporated by reference in its entirety, including all tables, figures and claims. One skilled in the art also recognizes that robotic instrumentation including but not limited to Beckman ACCESS®, Abbott AXSYM®, Roche ELECSYS®, Dade Behring STRATUS® systems are among the immunoassay analyzers that are capable of performing the immunoassays taught herein.

In some embodiment, methods for detecting an analyte in a biological sample comprises or consists essentially of enzyme-linked immunosorbent assay (ELISA), radioimmunoassays (RIAs), competitive binding assays, western blot, immunoprecipitation, and immunofluorescence using detection reagents such as an antibody or protein binding agents. In some embodiments, the biomarkers are analyzed using an immunoassay. The presence or amount of a marker is generally determined using antibodies specific for each marker and detecting specific binding. Specific immunological binding of the antibody to the marker can be detected directly or indirectly. Direct labels include fluorescent or luminescent tags, metals, dyes, radionuclides, and the like, attached to the antibody. Indirect labels include various enzymes well known in the art, such as alkaline phosphatase, horseradish peroxidase. Alternatively, a peptide can be detected in a biological sample from a subject by introducing into the sample a labeled anti-peptide antibody and other types of detection agent. For example, the antibody can be labeled with a detectable marker whose presence in the sample is detected by standard imaging techniques.

In some embodiments, the biomarkers are analyzed by measuring the marker RNA levels. Expression levels or abundance of the one or more makers can be determined by direct measurement of expression at the protein or mRNA level, for example by microarray analysis, quantitative PCR analysis, or RNA sequencing analysis. Alternatively, labeled antibody systems may be used to quantify target protein abundance in the cells, followed by immunofluorescence analysis, such as FISH analysis.

In some embodiments, the first biological sample is taken prior to administering a therapeutic regiment. In some embodiments, the second biological sample is taken after administering the therapeutic regiment. In some embodiments, a lack of elevated levels of KIM-1, IL-18, MCP-1, NGAL, UMOD, and/or YKL-40 in the second biological sample as compared to the first biological sample is indicative of continued kidney health and the therapeutic regimen should be continued. In various embodiments, an increase in the measured levels in the second biological sample as compared to the first biological sample is indicative of progression to chronic kidney disease (CKD) in the subject. In some embodiments, an increase in the measured levels in the second biological sample as compared to the first biological sample is indicative that the treatment for diabetes mellitus should be discontinued and/or the subject should be administered an alternative therapeutic regimen.

In some embodiments, the method of treatment further comprises or alternatively consists essentially of, or yet further consists of administering the therapeutic regimen to the subject if the levels of KIM-1, IL-18, MCP-1, NGAL, UMOD, and/or YKL-40 in the second biological sample are not elevated when compared to the first biological sample. In some embodiments, the therapeutic regimen causes a decrease in eGFR in the second biological sample when compared to the first biological sample.

In some embodiments, a subject having been diagnosed with diabetes mellitus will provide a urine sample prior to initiating a therapeutic regimen, and again after being on a therapeutic regimen for a predetermined amount of time. Various methods known in the art can also be utilized to determine the presence of a disease or condition described herein or to determine whether an immune response has been induced in a subject. Assessment of one or more biomarkers associated with a disease or condition, or for characterizing whether an immune response has been induced, can be performed by any appropriate method.

Exemplary predetermined amounts of time useful in the method so of the present disclosure include, but are not limited to, 1 month, 3 months, 6 months, 9 months, 1 year, 2 years, and 3 years, depending on the overall health of the subject. In some embodiments, the predetermined time is selected from 1 month, 3 months, 6 months, 9 months, 1 year, 2 years, or 3 years. In some embodiments, the method of treatment further comprise or alternatively consists essentially of, or yet further consists of administering the therapeutic regimen to the subject if the levels of KIM-1, IL-18, MCP-1, NGAL, UMOD, and/or YKL-40 in the second, third, fourth, sixth or seventh biological sample are not elevated when compared to the last biological sample tested. In some embodiments, the levels of KIM-1, IL-18, MCP-1, NGAL, UMOD, and/or YKL-40 in the biological sample of the subject is measured routinely during treatment. In some embodiments, the levels of KIM-1, IL-18, MCP-1, NGAL, UMOD, and/or YKL-40 in the biological sample of the subject is measured and compared to the last biological sample from the same subject at 1 month, 3 months, 6 months, 9 months, 1 year, 2 years, 3 years, or 4 years interval depending on the overall health of the subject.

### Therapeutic Regimen

In some embodiments, the therapeutic regimen is an antihypertensive regimen selected from of diuretics, renin-angiotensin system (RAS) antagonists, Angiotensin-converting enzyme (ACE) inhibitor, β-adrenergic blockers, a-adrenergic blockers, calcium channel blockers, or a combination thereof. In some embodiments, the therapeutic regimen is a diuretic. Diuretics control blood pressure by helping the body eliminate excess sodium (salt). In some embodiments, antihypertensive diuretics are selected from thiazide diuretics, potassium-sparing diuretics, poop diuretics, or combination diuretics. In some embodiments, thiazide diuretics comprise chlorthalidone, chlorothiazide, hydrochlorothiazide, indapamide, and metolazone. In some embodiments, potassium-sparing diuretics comprise amiloride hydrochloride, spironolactone, and triamterene. In some embodiments, loop diuretics comprise furosemide and bumetanide. In some embodiments, combination diuretics comprise amiloride hydrochloride and hydrochlorothiazide, spironolactone and hydrochlorothiazide, and triamterene and hydrochlorothiazide.

In some embodiments, the therapeutic regimen is a β-adrenergic blocker. β-adrenergic blockers control blood pressure by reducing the heart rate, the heart's workload and the heart's output of blood. In some embodiments, the β-adrenergic blocker is selected from acebutolol, atenolol, betaxolol, bisoprolol fumarate, carteolol hydrochloride, metoprolol tartrate, metoprolol succinate, nadolol, penbutolol sulfate, pindolol, propranolol hydrochloride, solotol hydrochloride, or timolol maleate. In some embodiments, the therapeutic regimen is a β-adrenergic blocker/diuretics combination such as hydrochlorothiazide and bisoprolol.

In some embodiments, the therapeutic regimen is an Angiotensin-converting enzyme (ACE) inhibitor. The renin-angiotensin-aldosterone axis is important in the maintenance of systemic blood pressure and sodium water homeostasis.³⁹ Renin, a proteolytic enzyme secreted by the juxtaglomerular apparatus of the kidney, cleaves angiotensinogen at the N terminus to form Angiotensin I, which is converted to Angiotensin II by ACE. ACE is a membrane-bound enzyme on the surface of endothelial cells, including lung, heart, brain and kidney. In the kidney, most of the intrarenal Angiotensin II is locally generated. Angiotensin II causes vasoconstriction (narrowing of arteries) thereby increasing arterial blood pressure. ACE inhibitors lower blood pressure by preventing the formation of angiotensin II and other metabolically active angiotensins. In some embodiments, the ACE inhibitor is selected from benazepril hydrochloride, captopril, enalapril maleate, fosinopril sodium, Lisinopril, moexipril, perindopril, quinapril hydrochloride, Ramipril, or trandolapril. The biological functions of Ang II are mediated by at least two pharmacologically distinct receptors, the Ang II type 1 (AT1) and Ang II type 2 (AT2) receptors.³⁹ AT1 receptors are abundantly expressed in cells of the renal glomeruli, tubules, vasculature and interstitial space. In some embodiments, the therapeutic regimen is an angiotensin II receptor blocker selected from candesartan, eprosartan mesylate, irbesarten, losartan potassium, telmisartan, or valsartan.

Calcium channel blockers relax and open up narrowed blood vessels, reduce heart rate and lower blood pressure by preventing the entry of calcium into heart muscle and preventing muscle hypercontraction. In some embodiments, the therapeutic regimen is a calcium channel blocker selected from amlodipine besylate, bepridil, diltiazem hydrochloride, felodipine, isradipine, nicardipine, nifedipine, nisoldipine, or verapamil hydrochloride.

Alpha-adrenergic blockers lower blood pressure by reducing the arteries' resistance and relaxing the muscle tone of the vascular walls. In some embodiments, the therapeutic regimen is an alpha-adrenergic blocker selected from doxazosin mesylate, prazosin hydrochloride, or terazosin hydrochloride. In some embodiments, the therapeutic regimen is an alpha-2-adrenergic receptor agonist such as methyldopa. Alpha-2-adrenergic receptor agonist reduces blood pressure by decreasing the activity of the sympathetic (adrenaline-producing) portion of the involuntary nervous system. In some embodiments, the therapeutic regimen is a combination of alpha- and beta-adrenergic blockers selected from carvedilol or labetalol hydrochloride.

In some embodiments, the therapeutic regimen is an antihypertensive therapeutic selected from chlorthalidone, chlorothiazide, hydrochlorothiazide, indapamide, metolazone, amiloride hydrochloride, spironolactone, triamterene, furosemide, bumetanide, amiloride hydrochloride, hydrochlorothiazide, spironolactone and hydrochlorothiazide, triamterene and hydrochlorothiazide, acebutolol, atenolol, betaxolol, bisoprolol fumarate, carteolol hydrochloride, metoprolol tartrate, metoprolol succinate, nadolol, penbutolol sulfate, pindolol, propranolol hydrochloride, solotol hydrochloride, or timolol maleate, hydrochlorothiazide and bisoprolol, benazepril hydrochloride, captopril, enalapril maleate, fosinopril sodium, Lisinopril, moexipril, perindopril, quinapril hydrochloride, Ramipril, trandolapril candesartan, eprosartan mesylate, irbesarten, losartan potassium, telmisartan, valsartan, amlodipine besylate, bepridil, diltiazem hydrochloride, felodipine, isradipine, nicardipine, nifedipine, nisoldipine, verapamil hydrochloride, doxazosin mesylate, prazosin hydrochloride, or terazosin hydrochloride, carvedilol, labetalol hydrochloride alpha methyldopa, clonidine hydrochloride, guanabenz acetate, guanfacine hydrochloride, guanadrel, guanethidine monosulfate, reserpine, hydralazine hydrochloride, or minoxidil.

Hypertension, which is also known as high blood pressure is defined as a blood pressure at or above 130/80 mm Hg. Stage 2 hypertension is defined as a blood pressure at or above 140/90 mm Hg. Normal blood pressure is defined as a blood pressure at or below <120/80 mm Hg, and elevated blood pressure is defined as a blood pressure at or below <120-129/80 mm Hg. Having hypertension puts you at risk for heart disease and stroke, which are leading causes of death in the United States. Nearly 45% of adults in the United States have hypertension. Hypertension is common and a significant risk factor for cardiovascular disease (CVD).¹⁻³ A number of clinical trials and meta-analyses have demonstrated that treatment of hypertension lowers risk for CVD and all-cause mortality.⁴⁻⁶ However, the effects of blood pressure (BP) lowering on chronic kidney disease (CKD) progression are less clear⁷⁻¹⁰ because treating to lower BP targets results in higher risk for acute kidney injury (AKI) and more rapid loss of estimated glomerular filtration rate (eGFR).^{8,11,12} The risks for both AKI and eGFR loss may be particularly concerning in patients with prevalent CKD because they have lower eGFRs at baseline and therefore may be least able to tolerate additional kidney insults. Balancing the risks and benefits, the appropriate BP target in patients with CKD remains an area of controversy.

The “estimated glomerular filtration rate (eGFR)” is derived at by screening serum creatinine values based on e.g., the Chronic Kidney Disease Epidemiology Collaboration (CKD-EPI) equation, the Cockcroft-Gault formula or the Modification of Diet in Renal Disease (MDRD) formula, which are all known in the art. The GFR is determined by injecting inulin into the plasma. Since inulin is neither reabsorbed nor secreted by the kidney after glomerular filtration, its rate of excretion is directly proportional to the rate of filtration of water and solutes across the glomerular filter. In some embodiments, GFR measurement involves isotopic such as chromium-51 (⁵¹Cr)-EDTA, iodine-125 (¹²⁵I)-iothalamate or technetium-99m diethylenetriaminepentaacetic acid ([^{99m}Tc]DTPA). GFR measurement involves non-isotopic such as iohexol or iothalamate. In a healthy subject, the GFR is between 90-125 mL/min/1.73 m². In some embodiments, the GFR of a normal subject is 100-125 mL/min/1.73 m².

In some embodiments, the antihypertensive treatment is maintained if the levels of KIM-1, IL-18, MCP-1, NGAL, UMOD, and/or YKL-40 in subsequent biological samples are stable when compared to the first or previous biological sample and in conjunction with a rapid loss of eGFR. In some embodiments, the antihypertensive treatment is discontinued if the levels of KIM-1, IL-18, MCP-1, NGAL, UMOD, and/or YKL-40 in the second or subsequent biological samples are elevated when compared to the first or previous biological sample in conjunction with a rapid loss of eGFR.

In some embodiments, the antihypertensive treatment induced-fall of eGFR is at least 11% reduction, and the levels of KIM-1, IL-18, MCP-1, NGAL, UMOD, and/or YKL-40 in subsequent biological samples are stable when compared to the first or previous biological sample. In some embodiments, the eGFR in the second biological sample is less than 40 ml/min/1.73m² and the levels of KIM-1, IL-18, MCP-1, NGAL, UMOD, and/or YKL-40 in subsequent biological samples are stable when compared to the first or previous biological samples. In some embodiments, the method further comprises discontinuing the therapeutic regiment if the levels of KIM-1, IL-18, MCP-1, NGAL, UMOD, and/or YKL-40 in the second biological sample are elevated when compared to the first biological sample and the eGFR is reduced by at least about 11%, or the eGFR in the second biological sample is less than 40 ml/min/1.73 m².

In some embodiments, the subject is at risk of renal impairment or chronic kidney disease (CKD). In some embodiments, the CKD may be any stage, including, for example, Stage 1, Stage 2, Stage 3, Stage 4, or Stage 5 (also known as established CKD, end-stage renal disease (ESRD), chronic kidney failure (CKF), or chronic renal failure (CRF)). In some embodiment, the CKD may be caused by hypertension treatment and additionally, any one of a number of factors, including, but not limited to, acute kidney injury, causes of acute kidney injury, Type 1 and Type 2 diabetes mellitus leading to diabetic nephropathy, high blood pressure (hypertension), glomerulonephritis (inflammation and damage of the filtration system of the kidneys), polycystic kidney disease, use (e.g., regular and over long durations of time) of analgesics (e.g., acetaminophen, ibuprofen) leading to analgesic nephropathy, atherosclerosis leading to ischemic nephropathy, obstruction of the flow of urine by stones, an enlarged prostate strictures (narrowings), HIV infection, sickle cell disease, illicit drug (e.g., heroin, cocaine) abuse, amyloidosis, kidney stones, chronic kidney infections, and certain cancers. In some embodiments, a subject is at risk of renal impairment or chronic kidney disease (CKD), when the eGFR falls below 60 ml/min/1.73 m².

### Predicting an Adverse Health Condition

In another aspect, the present disclosure provides methods for predicting an adverse health condition in a subject undergoing a therapeutic regimen for diabetes mellitus. The method includes measuring the levels of one or more of: α₁-microglobulin (A1M), β₂-microglobulin (B2M), kidney injury molecule 1 (KIM-1), interleukin 18 (IL-18), monocyte chemoattractant protein 1 (MCP-1), neutrophil gelatinase-associated lipocalin (NGAL), uromodulin (UMOD), and/or human cartilage glycoprotein 39 (YKL-40) in a first biological sample from the subject prior to beginning the therapeutic regimen; commencing the therapeutic regimen; and measuring the levels of one or more of A1M, B2M, KIM-1, IL-18, MCP-1, NGAL, UMOD, and/or YKL-40 in a second biological sample from the subject obtained after a predetermined time after commencing the therapeutic regimen. In various embodiments, elevated levels of KIM-1, IL-18, MCP-1, NGAL, UMOD, and/or YKL-40 in the second biological sample as compared to the first biological sample is indicative of progression to chronic kidney disease (CKD) in the subject and the therapeutic regimen should be discontinued and/or the subject should be administered an alternative therapeutic regimen. In various embodiments, a lack of elevated levels of KIM-1, IL-18,-MCP-1, NGAL, UMOD, and/or YKL-40 in the second biological sample as compared to the first biological sample is indicative of continued kidney health and the therapeutic regimen should be continued in view of the long-term benefits associated with the therapeutic regimen.

In various embodiments, a subject having been diagnosed with diabetes mellitus will provide a urine sample prior to imitating SGLT2 inhibitor therapy, and again after being on SGLT2 inhibitors after a predetermined amount of time. Exemplary predetermined amounts of time useful in the method so of the present disclosure include, but are not limited to, 1 month, 3 months, 6 months, 9 months, 1 year, 2 years, and 3 years, depending on the overall health of the subject.

As such, the diagnostic panel of eight markers would be measured at multiple time-points to determine the degree of change (if any) in response to drug therapy to differentiate hemodynamic change versus intrinsic kidney damage in the subject. The results of each biomarker, and a laboratory interpretation will be provided back to the medical practitioner to determine if the subject should discontinue use of SGLT2 inhibitor therapy to improve kidney health.

In some embodiments, diabetes is associated with vascular diseases. In some embodiments, the vascular disease is chronic kidney disease (CKD). Chronic kidney disease is defined as a reduced glomerular filtration rate, increased urinary albumin excretion, or both. In some embodiments, complications of diabetes include increased all-cause and cardiovascular mortality, kidney-disease progression, acute kidney injury, cognitive decline, anemia, mineral and bone disorders, and fractures. In some embodiments, CKD in diabetes, is also referred to as diabetic kidney disease (DKD). In some embodiments, the symptoms of DKD are persistent microalbuminuria or decreased glomerular filtration rate (GFR). Standard treatment for DKD is a reduction of blood pressure by interfering with the rennin/angiotension/aldosterone systems (RAAS). Initial studies of the use of SGLT2 inhibitors in patients with chronic kidney disease (CKD) showed that they were associated with an early and dose-dependent increase in serum creatinine or blood urea nitrogen (BUN) levels and a decrease in estimated glomerular filtration rate (eGFR).²⁹ Because of these reports, the US Food and Drug Administration (FDA) strengthened and maintains an existing warning about the risk of acute kidney injury for canagliflozin and dapagliflozin. In particular, the risk of adverse renal events was increased with the use of dapagliflozin or canagliflozin as compared with placebo.²⁹ Dapagliflozin is associated with an increased incidence of renal impairment or failure and creatinine increase or eGFR decrease in patients with Type 2 diabetes, in elderly patients or patients with extant renal impairment. Generally, the evidence from individual randomized trials has been inconsistent regarding the possible adverse effects of SGLT2 inhibitors on renal outcomes. Accordingly, the present disclosure provides a method for determining whether abnormal changes in eGFR or creatinine during SGLT2 therapy might reflect a temporary and reversible change in renal function caused by haemodynamic changes related to osmotic diuresis, reduction in blood pressure, or altered intrarenal haemodynamics, or kidney injury.

In some embodiments, a method of determining whether a subject is amenable to treatment with an inhibitor of SGLT2 for diabetes mellitus. The method can be performed, for example, by measuring the levels of KIM-1, IL-18, MCP-1, NGAL, UMOD, and/or YKL-40 in a biological sample of a subject to be treated, and determining whether the levels are elevated or abnormally elevated as compared to the levels of a corresponding normal sample and/or as compared to levels in a sample after therapy has commenced. Detection of elevated or abnormally elevated levels of KIM-1, IL-18, MCP-1, NGAL, UMOD, and/or YKL-40 in the sample as compared to the levels in a corresponding normal sample, or as compared to levels after therapy has commenced indicates that the subject should discontinue treatment due to progression to CKD. In some embodiments, one or more of the methods described herein further comprise, or consists essentially of, or yet further consists of, a diagnostic step.

In another aspect, the present disclosure provides methods for predicting an adverse health condition in a subject undergoing a therapeutic regimen for diabetes mellitus. The method includes measuring the levels one or more of α₁-microglobulin (A1M), β₂-microglobulin (B2M), kidney injury molecule 1 (KIM-1), interleukin 18 (IL-18), monocyte chemoattractant protein 1 (MCP-1), neutrophil gelatinase-associated lipocalin (NGAL), uromodulin (UMOD), or human cartilage glycoprotein 39 (YKL-40) in a first biological sample from the subject prior to beginning the therapeutic regimen; commencing the therapeutic regimen; and measuring the levels of one or more of A1M, B2M, KIM-1, IL-18, MCP-1, NGAL, UMOD, or YKL-40 in a second biological sample from the subject obtained after a predetermined time after commencing the therapeutic regimen. In various embodiments, elevated levels of KIM-1, IL-18, MCP-1, NGAL, UMOD, or YKL-40 in the second biological sample as compared to the first biological sample is indicative of progression to chronic kidney disease (CKD) in the subject and the therapeutic regimen should be discontinued and/or the subject should be administered an alternative therapeutic regimen. In various embodiments, a lack of elevated levels of KIM-1, IL-18, MCP-1, NGAL, UMOD, or YKL-40 in the second biological sample as compared to the first biological sample is indicative of continued kidney health and the therapeutic regimen should be continued in view of the long-term benefits associated with the therapeutic regimen.

### Kits

The present disclosure also contemplates commercial kits and articles of manufacture specific for performing the assays and methods described herein. In another aspect, the present disclosure provides a kit or article of manufacture comprising, consisting essentially of, or further yet consisting of: (i) reagents specific to measure the levels of one or more of A1M, B2M, KIM-1, IL-18, MCP-1, NGAL, UMOD, or YKL-40 in a biological sample obtained from a subject; and (ii) instructions for monitoring kidney health in the subject undergoing treatment for diabetes mellitus. In some embodiments, the kit comprises instructions for predicting an adverse health condition in a subject undergoing treatment with a therapeutic regimen that induces renal impairment or chronic kidney disease. In some embodiments, the therapeutic regimen is for diabetes mellitus. In some embodiments, the therapeutic regimen is for hypertension.

As used herein, a kit or article of manufacture described herein include a carrier, package, or container that is compartmentalized to receive one or more containers such as vials, tubes, and the like, each of the container(s) comprising, or consisting essentially of, or yet further consisting of, one of the separate elements to be used in a method described herein. Suitable containers include, for example, bottles, vials, syringes, and test tubes. In one embodiment, the containers are formed from a variety of materials such as glass or plastic.

The articles of manufacture provided herein contain packaging materials. Examples of pharmaceutical packaging materials include, but are not limited to, blister packs, bottles, tubes, bags, containers, bottles, and any packaging material suitable for a selected formulation and intended mode of administration and treatment. A kit typically includes labels listing contents and/or instructions for use, and package inserts with instructions for use. A set of instructions will also typically be included.

The following examples are intended to illustrate but not limit the disclosure.

### EXAMPLES

The examples herein are provided to illustrate advantages of the present technology and to further assist a person of ordinary skill in the art with preparing and/or using the compounds of the present technology. The examples herein are also presented in order to more fully illustrate the preferred aspects of the present technology. The examples should in no way be construed as limiting the scope of the present technology. The examples can include or incorporate any of the variations, aspects, or embodiments of the present technology described above. The variations, aspects, or embodiments described above may also further each include or incorporate the variations of any or all other variations, aspects or embodiments of the present technology.

### Example 1: Material and Methods

Study Design. The trial design and results for the primary CVD end point of SPRINT have been reported previously.^{13, 18} Briefly, SPRINT is an open-label clinical trial that randomly assigned persons with SBP≥130 mm Hg and high risk for CVD events to an SBP target of <120 mm Hg (“intensive”) versus <140 mm Hg (“standard”).¹⁸ Participants were recruited from 102 centers in the United States and Puerto Rico. Inclusion criteria required age of 50 years and older, SBP of 130 to 180 mm Hg, and increased risk for CVD events (prior clinical or subclinical CVD other than stroke, 10-year risk for CVD≥15% based on the Framingham risk score, CKD defined as eGFR of 20-59 mL/min/1.73 m², or age ≥75 years). Major exclusion criteria included diabetes mellitus, proteinuria with protein excretion >1 g/d, polycystic kidney disease, prior stroke or transient ischemic attack, symptomatic heart failure, or left ventricular ejection fraction <35%. A total of 9,361 participants were enrolled and all participants provided written informed consent.

Serum cystatin C was measured in all SPRINT participants, and a subset with eGFRs <60 mL/min/1.73 m² was defined using the CKD-EPI (CKD Epidemiology Collaboration) creatinine-cystatin C equation. From the 2,646 participants with baseline eGFRs <60 mL/min/1.73 m², 1,000 individuals were selected using simple random sampling for participation. Twenty-two individuals had unavailable urine specimens and were excluded from further analysis. Thus, the final analytic sample included 978 participants with CKD at baseline.

In SPRINT, participants were randomly assigned in a 1:1 ratio to the intensive or standard arm. Antihypertensive regimens were adjusted to maintain SBP according to the randomly assigned BP groups.¹⁸ Participants attended visits monthly for the first 3 months and then every 3 months thereafter, and clinical and laboratory data were obtained at these visits. Venous blood and urine specimens were immediately processed, shipped overnight on ice packs, and stored at −80° C. at a central laboratory for use in future studies. eGFR decline and changes in urinary albumin-creatinine ratio (ACR) in the CKD subgroup have previously reported,¹⁴ and showed that random assignment to the intensive group was associated with more rapid decline in eGFR and a decrease in urinary ACR relative to the standard group among participants with CKD.

Kidney Tubule Biomarker Measurements. Urinary specimens from the baseline, 12-month, and 48-month SPRINT visits were stored at −80° C. until thawing for kidney tubule cell damage biomarker measurements. All specimens were thawed at once and measurements were completed en bloc to minimize any influence of analytic drift on longitudinal changes in biomarkers.

Eight distinct urinary markers were chosen because they reflect aspects of kidney tubule biology, including tubule function, injury, inflammation, and repair. The eight urinary markers include tubule function (β2-microglobulin (B2M); α1-microglobulin (A1M); and uromodulin (UMOD)), injury (interleukin 18 (IL-18); kidney injury molecule 1 (KIM-1); and neutrophil gelatinase-associated lipocalin (NGAL)), inflammation (monocyte chemoattractant protein 1 (MCP-1)), and repair (human cartilage glycoprotein 39 (YKL-40)) and are known to be associated with CKD progression.¹⁹⁻²⁴

B2M and A1M are low-molecular-weight proteins that are freely filtered at the glomerulus and then reabsorbed by the proximal tubule. Higher levels of these proteins in urine are associated with kidney function decline among persons infected with human immunodeficiency virus (HIV).¹⁹ UMOD is a 95-kDa glycoprotein synthesized exclusively by kidney tubules. Higher UMOD levels are associated with kidney size and eGFR, and lower UMOD levels are associated with CKD progression.²⁰ IL-18, KIM-1, and NGAL are markers of tubular injury, with urine levels increasing by several-fold in response to ischemic or inflammatory kidney injury.^{21,22} MCP-1 is a chemokine that attracts macrophages to the site of injury, and strong associations of this marker have been shown with CKD progression in kidney transplant recipients.²³ Finally, YKL-40 functions as a mediator of the reparative response to tubular injury.²⁴ Taken together, the selected urinary markers measure the interlinked axes of inflammation, tubular injury and atrophy, and tubulointerstitial fibrosis, which are hallmarks of progressive CKD.

Urinary biomarkers were measured centrally at the Laboratory for Clinical Biochemistry Research at the University of Vermont. Most urinary biomarkers (all except A1M) were measured using multiplex assays on a MESO Scale Diagnostics (MSD) platform. Urinary YKL-40, IL-18, MCP-1, and KIM-1 measures were conducted together on a 4-plex assay. Analytic ranges were 10 to 500,000 ng/mL, 2 to 10,000 ng/mL, 3 to 10,000 pg/mL, and 4 to 200,000 pg/ mL for YKL-40, IL-18, MCP-1, and KIM-1, respectively. Interassay coefficients of variation (CVs) ranged across the analytic range from 6.5% to 11.1%, 4.9% to 13.7%, 7.1% to 12.0%, and 6.1% to 13.0%, respectively.

A second 3-plex MSD assay was used to measure urinary B2M, UMOD, and NGAL; for these, analytic ranges were 1.2 to 5,020 ng/mL, 0.6 to 2,510 ng/mL, and 6 to 251,000 ng/mL, respectively, and interassay CVs were 15% to 16%, 13% to 16%, and 11% to 19%. For urinary A1M, a Siemens nephelometric assay was used with a detectable range from 5 to 480 mg/L and interassay CVs ranging from 3.5% to 8.8% across the analytic range. Each marker was measured twice in each urine specimen and results were averaged to improve precision. Urine creatinine was measured using an enzymatic procedure (Roche), and urine albumin, using a nephelometric method (Siemens).¹⁴

Statistical Analysis. Descriptive statistics were computed and participant characteristics were compared across randomization arms at baseline using χ² test for categorical variables and t test for continuous variables or Kruskal-Wallis test when warranted due to skewness. In instances in which urine biomarker levels were below the limit of assay detection, the value of the lower limit was imputed. Distributions of outcomes were evaluated for normality and logy transformations were used to correct urine biomarker levels for positive skewness. To make presentation of all results consistent, log₂ transformation of eGFR was also used when modeling it as an outcome. Changes in outcomes over time between randomization arms were evaluated using mixed-effects linear models in a repeated-measures layout with unstructured 3×3 variance-covariance matrix to account for within-subject correlation among the 3 measured time points (baseline, 12 months, and 48 months).

All analyses were performed using the intention-to-treat approach. Exploratory analyses of urinary biomarkers demonstrated that model fit was best when terms adjusting for linear and quadratic urine creatinine concentration were included. Thus, all models for those outcomes (with the exception of ACR) included both linear and quadratic terms for logarithmically transformed (base 2) urine creatinine as time-varying covariates to account for differences in urine tonicity. Time (baseline, month 12, or month 48 follow-up) was treated as a class effect in all models. The difference in geometric least squares means of log-transformed urinary biomarkers between the intensive and standard BP arms was determined and back-transformation provided the ratio of geometric means and the related 95% confidence intervals (CIs).

For additional insights, 1- and 4-year changes in urinary biomarker levels were evaluated and their associations with quintiles of change in eGFR were compared using linear analysis of covariance models that adjusted for concurrently measured urine creatinine. This approach allowed for the determination of whether the magnitude of change in urine biomarker levels mirrored concurrent changes in eGFRs. Quintiles of eGFR change were defined based on the distribution observed in the intensive BP arm and applied the resulting cut-points to the standard BP arm for consistency. Similarly, participants were stratified into quintiles of observed changes in SBP during the trial within the intensive BP arm and compared the magnitude of changes in urine biomarker levels across quintiles, again using linear analysis of covariance models.

### Example 2: Distribution of All Measurements Were Similar Between Standard Arm and Intensive Arm

A total of 978 SPRINT participants had baseline eGFRs<60 mL/min/1.73 m². The mean age was 72±9 years, 60% were men, 66% were non-Hispanic whites, and 39% had a history of CVD. At baseline, the median estimated glomerular filtration rate (eGFR) was 48 (interquartile range [IQR], 40-54) mL/min/1.73 m², the median urinary albumin-creatinine ratio (ACR) was 15 (IQR, 7-52) mg/g, and the mean systolic BP was 139±16 and the mean diastolic BP was 75 ±12 mm Hg. The mean number of antihypertensive medications at baseline was 2.0±1.0.

Among the 978 SPRINT participants, Five hundred nineteen (519) participants were randomly assigned to the intensive BP arm (<120 mmHg), and 459 were randomly assigned to the standard BP arm (<140 mmHg). Baseline demographic, clinical, and laboratory characteristics are displayed by randomly assigned treatment arm in Table 1. As shown in table 1, the distributions of all measurements were similar across arms except for serum triglyceride levels, which were lower in the intensive BP arm (124±61 vs 135±92 mg/dL; P=0.04).

TABLE 1
Baseline Characteristics of Participants
by Randomized Treatment Arm

| | Intensive BP Arm | | Standard BP Arm | | |
|---|---|---|---|---|---|
| | (n = 519) | | (n = 459) | | P |
| Age, y | 72 ± 9 | | 72 ± 9 | | 0.9 |
| Male sex | 304 | (59%) | 280 | (61%) | 0.4 |
| White | 345 | (67%) | 302 | (66%) | 0.8 |
| Education: some college | 376 | (73%) | 318 | (69%) | 0.3 |
| or greater | | | | | |
| Current smoker | 232 | (45%) | 205 | (45%) | 0.9 |
| History of heart disease | 212 | (41%) | 166 | (36%) | 0.1 |
| History of PVD | 42 | (8%) | 44 | (10%) | 0.4 |
| SBP, mm Hg | 139 ± 16 | | 140 ± 17 | | 0.4 |
| DBP, mm Hg | 75 ± 12 | | 74 ± 12 | | 0.9 |
| No. of BP medications | 2 ± 1 | | 2 ± 1 | | 0.5 |
| Treated by diuretic | 199 | (38%) | 269 | (59%) | 0.3 |
| Treated by ARB or ACEi | 253 | (49%) | 229 | (50%) | 0.7 |
| Total cholesterol, mg/dL | 185 ± 41 | | 182 ± 39 | | 0.3 |
| HDL cholesterol, mg/dL | 52 ± 14 | | 52 ± 15 | | 0.9 |
| Triglycerides, mg/dL | 124 ± 61 | | 135 ± 92 | | 0.04 |
| Body mass index, kg/m² | 30 ± 6 | | 29 ± 6 | | 0.09 |
| eGFR, mL/min/1.73 m² | 48 | [39-54] | 48 | [41-54] | 0.7 |
| Urinary ACR, mg/g | 15 | [7-51] | 16 | [7-56] | 0.9^{a} |

Note:
Values for continuous variables are given as mean ± standard deviation or median [interquartile range]; those for categorical variables, as count (percentage).
Abbreviations: ACEi, angiotensin-converting enzyme inhibitor; ACR, albumin-creatinine ratio; ARB, angiotensin II receptor blocker; BP, blood pressure; DBP, diastolic blood pressure; eGFR, estimated glomerular filtration rate; HDL, high-density lipoprotein; PVD, peripheral vascular disease; SBP, systolic blood pressure.
^{a}P value from Kruskal-Wallis test for difference in median values.

A comparison of baseline factors among participants with chronic kidney disease (CKD) who were sampled versus those who were not sampled showed similar results (Table 2). In that comparison, participant that were sampled appeared slightly younger, had slightly lower Framingham risk scores, and had slightly lower eGFRs at baseline.

TABLE 2
Baseline characteristics of participants
by samples versus nonsampled status.

| | Sampled | Non-sampled |
|---|---|---|
| Variables | (n = 955) | (n = 1546) |
| Randomization arm, n | | |
| (%) Intensive BP | 501 (52.5) | 776 (50.2) |
| Standard BP | 454 (47.5) | 770 (49.8) |
| Age, years mean ± SD | 71.9 ± 8.7 | 73.9 ± 9.3 |
| Race, white n (%) | 630 (66.0) | 1018 (65.9) |
| Gender, female n (%) | 390 (40.8) | 617 (39.9) |
| History of heart disease n (%) | 251 (26.3) | 376 (24.3) |
| 10-year Framingham risk mean ± SD | 27.4 ± 14.8 | 28.9 ± 14.5 |
| SBP, mmHg mean ± SD | 139.3 ± 16.5 | 140.0 ± 16.3 |
| DBP, mmHg mean ± SD | 74.4 ± 12.2 | 74.4 ± 12.5 |
| eGFR ml/min/1.73 m2 mean ± SD | 44.1 ± 10.4 | 46.8 ± 10.5 |

Abbreviations: BP; blood pressure, SD; standard deviation, SBP; systolic blood pressure, DBP; diastolic blood pressure, eGFR; estimated glomerular filtration rate.

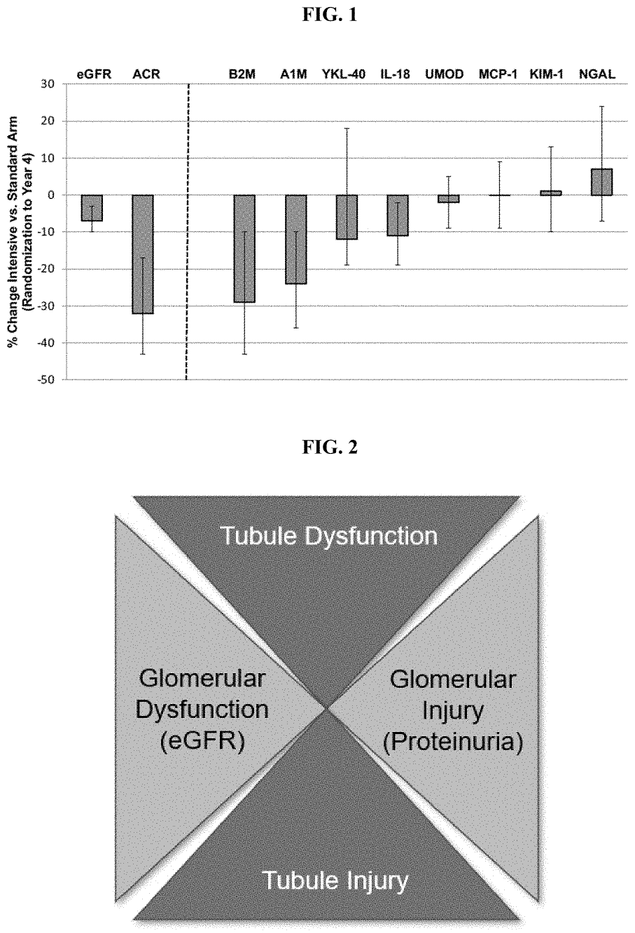

Baseline concentrations of each of the eight urinary tubule biomarkers (B2M, A1M, UMOD, IL-18, KIM-1, NGAL, MCP-1, and YKL-40) were also similar across the 2 arms. However, at the 1 year, eGFR was 7% lower and ACR was 32% lower among participants randomly assigned to intensive BP compared to the standard arm in the subset included in this analysis (Table 3; FIG. 1). In addition, none of the eight urinary tubular marker levels were higher in the intensive versus standard arm at year 1.

TABLE 3
Results of Generalized Linear Mixed-Effects Models of Effects of Intensive versus Standard BP
Control on eGFR, Albuminuria, and Urinary Tubular Markers in Participants With CKD in SPRINT

| | | | |
|---|---|---|---|
| Intensive BP Arm | Standard BP Arm | Ratio^{a} | P |

eGFR, mL/min/1.73 m²

| | | | | |
|---|---|---|---|---|
| Baseline | n = 519; 44.8 (44.0, 45.7) | n = 459; 45.2 (44.3, 46.2) | ↓1% (↓4%, | 0.5 |
| | | | ↑2%) | |
| Year 1 | n = 512; 43.1 (42.0, 44.2) | n = 455; 46.3 (45.0, 47.5) | ↓7% (↓10%, | <0.001 |
| | | | ↓3%) | |
| Year 4 | n = 494; 41.4 (40.0, 42.8) | n = 425; 44.6 (43.1, 46.3) | ↓7% (↓12%, | 0.002 |
| | | | ↓3%) | |

ACR, mg/g

| | | | | |
|---|---|---|---|---|
| Baseline | n = 507; 21.9 (19.3, 24.9) | n = 452; 22.0 (19.3, 25.2) | ↔0% (↓17%, | 0.9 |
| | | | ↑20%) | |
| Year 1 | n = 485; 17.9 (15.7, 20.4) | n = 434; 26.2 (22.8, 30.0) | ↓32% (↓43%, | <0.001 |
| | | | ↓17%) | |
| Year 4 | n = 483; 24.8 (21.6, 28.5) | n = 421; 35.8 (30.9, 41.4) | ↓31% (↓43%, | <0.001 |
| | | | ↓15%) | |

Log₂ B2M/cr, ng/g

| | | | | |
|---|---|---|---|---|
| Baseline | n = 519; 101.9 (87.5, 118.8) | n = 459; 105.5 (89.8, 123.9) | ↑3% (↓23%, | 0.7 |
| | | | ↑21%) | |
| Year 1 | n = 513; 104.4 (89.0, 122.4) | n = 454; 146.3 (123.5, 173.2) | ↓29% (↓43%, | 0.005 |
| | | | ↓10%) | |
| Year 4 | n = 461; 137.0 (117.0, 160.5) | n = 401; 155.8 (131.6, 184.4) | ↓12% (↓30%, | 0.3 |
| | | | ↑11%) | |

Log₂ A1M/cr, mg/g

| | | | | |
|---|---|---|---|---|
| Baseline | n = 519; 10.4 (9.3, 11.6) | n = 459; 10.9 (9.7, 12.3) | ↓5% (↓19%, | 0.5 |
| | | | ↑11%) | |
| Year 1 | n = 513; 8.2 (7.3, 9.2) | n = 454; 10.8 (9.5, 12.3) | ↓24% (↓36%, | 0.002 |
| | | | ↓10%) | |
| Year 4 | n = 461; 9.4 (8.2, 10.7) | n = 401; 10.6 (9.2, 12.2) | ↓12% (↓27%, | 0.2 |
| | | | ↑7%) | |

Log₂ YKL-40/cr. ng/g

| | | | | |
|---|---|---|---|---|
| Baseline | n = 519; 442.8 (388.3, 505.0) | n = 459; 431.5 (375.2, 496.3) | ↑3% (↓15%, | 0.8 |
| | | | ↑24%) | |
| Year 1 | n = 513; 474.1 (416.2, 540.1) | n = 454; 485.0 (422.2, 557.0) | ↓12% (↓19%, | 0.8 |
| | | | ↑18%) | |
| Year 4 | n = 461; 594.1 (515.1, 685.3) | n = 401; 660.6 (567.0, 769.6) | ↓10% (↓27%, | 0.3 |
| | | | ↑11%) | |

Log₂ IL-18/cr, ng/g

| | | | | |
|---|---|---|---|---|
| Baseline | n = 519; 26.8 (25.0, 28.6) | n = 459; 28.3 (26.4, 30.4) | ↓6% (%↓14%, | 0.3 |
| | | | ↑4%) | |
| Year 1 | n = 513; 28.3 (26.5, 30.2) | n = 454; 31.9 (29.8, 34.2) | ↓11% (↓19%, | 0.01^{b} |
| | | | ↓2%) | |
| Year 4 | n = 461; 30.2 (28.3, 32.2) | n = 401; 31.0 (28.9, 33.2) | ↓3% (↓11%, | 0.6 |
| | | | ↑7%) | |

Log2 UMOD/cr, ng/g

| | | | | |
|---|---|---|---|---|
| Baseline | n = 519; 6,366.2 (6,038.2, 6,711.9) | n = 459; 5,993.4 (5,667.1, 6,338.4) | ↑6% (↓2%, | 0.1 |
| | | | ↑15%) | |
| Year 1 | n = 513; 6,180.9 (5,879.8, 6,497.5) | n = 454; 6,325.2 (5,997.8, 6,670.5) | ↓2% (↓9%, | 0.5 |
| | | | ↑5%) | |
| Year 4 | n = 461; 5,079.2 (4,796.0, 5,739.1) | n = 401; 5,266.3 (4,951.3, 5,601.3) | ↓4% (↓11%, | 0.4 |
| | | | ↑5%) | |

Log₂ MCP-1/cr, pg/g

| | | | | |
|---|---|---|---|---|
| Baseline | n = 519; 152.4 (143.3, 162.0) | n = 459; 147.8 (138.5, 157.8) | ↑3% (↓6%, | 0.5 |
| | | | ↑13%) | |
| Year 1 | n = 513; 151.6 (142.9, 160.9) | n = 454; 152.1 (142.7, 162.0) | ↔0% (↓9%, | 0.9 |
| | | | ↑9%) | |
| Year 4 | n = 461; 155.4 (146.2, 165.2) | n = 401; 151.2 (141.7, 161.5) | ↑3% (↓6%, | 0.6 |
| | | | ↑12%) | |

Log₂ KIM-1/cr, pg/g

| | | | | |
|---|---|---|---|---|
| Baseline | n = 519; 673.6 (626.1, 724.6) | n = 459; 632.1 (584.8, 683.1) | ↑7% (↓4%, | 0.2 |
| | | | ↑19%) | |
| Year 1 | n = 513; 694.5 (644.3, 748.7) | n = 454; 688.3 (635.5, 745.5) | ↑1% (↓10%, | 0.9 |
| | | | ↑13%) | |
| Year 4 | n = 461; 820.2 (762.7, 882.1) | n = 401; 771.1 (713.5, 833.4) | ↑6% (↓4%, | 0.3 |
| | | | ↑18%) | |

Log₂ NGAL/cr, ng/g

| | | | | |
|---|---|---|---|---|
| Baseline | n = 519; 29.9 (27.1, 32.9) | n = 459; 28.3 (25.6, 31.4) | ↑6% (↓8%, | 0.5 |
| | | | ↑22%) | |
| Year 1 | n = 513; 33.9 (30.7, 37.5) | n = 454; 31.6 (28.4, 35.2) | ↑7% (↓7%, | 0.4 |
| | | | ↑24%) | |
| Year 4 | n = 461; 36.2 (32.7, 40.1) | n = 401; 32.9 (29.5, 36.6) | ↑10% (↓5%, | 0.2 |
| | | | ↑28%) | |

Note:
Values are given as number of patients; least-squares mean (95% CI).
Abbreviations: A1M, α1-microglobulin; ACR, albumin-creatinine ratio; B2M, β2-microglobulin; BP, blood pressure; CKD, chronic kidney disease; CI, confidence interval; Cr, creatinine; eGFR, estimated glomerular filtration rate; IL-18, interleukin 18; KIM-1, kidney injury molecule 1; MCP-1, monocyte chemoattractant protein 1; NGAL, neutrophil gelatinase-associated lipocalin; SPRINT, Systolic Blood Pressure Intervention Trial; UMOD, uromodulin; YKL-40, human cartilage glycoprotein 39.
^{a}Ratio of intensive arm to standard arm least-squares means. All urinary biomarkers were adjusted for linear and quadratic urine creatinine concentrations. Values in parentheses are 95% CIs.
^{b}IL-18: overall, the test for the treatment × visit interaction is not significant (P = 0.2).

Using an omnibus test to compare differences in urinary biomarker levels across baseline, year 1, and year 4, statistically significant differences were detected for two biomarkers: B2M (P=0.03) and A1M (P=0.01) across study years. Comparing differences at year 1, B2M level was 29% (95% CI, 10%-43%) lower, and A1M level was 24% (95% CI, 10%-36%) lower in the intensive arm relative to the standard arm. Although the omnibus P value did not reach statistical significance across years for urinary IL-18 level (P=0.2), a similar difference was observed at year 1, in which IL-18 concentrations were 11% (95% CI, 2%-19%) lower in the intensive versus standard arm at year 1 (P=0.01). At year 4, differences in eGFRs and urinary ACRs across BP arms were similar to year 1 (7% and 31% lower in the intensive vs standard arm, respectively). However, statistically significant differences in any urinary tubule marker level across arms at year 4 were not observed.

Change in urinary ACR and urine tubule marker levels in participants with extremes of eGFR changes at years 1 and 4 in both the intensive and standard BP arms were also evaluated. In the intensive arm, persons who experienced decreases in eGFRs among the highest quintile had the largest reductions in urinary ACR, B2M, A1M, and IL-18 levels at year 1. Evaluation of heterogeneity across eGFR categories was statistically significant for ACR, and IL-18 (all P<0.01) and approached statistical significance for B2M (P=0.07; Table 4). As shown in Table 5, at year 4, changes in urine biomarker levels were not related to the magnitude of change in eGFR in either treatment arm.

TABLE 4
Comparison of 1- and 4-Year Changes in Albuminuria and Urinary Tubule Biomarkers Stratified
by Quintile of eGFR Change and Randomization Arm Among Participants With CKD in SPRINT

| | | | | | |
|---|---|---|---|---|---|
| ΔeGFR Q1 | ΔeGFR Q2 | ΔeGFR Q3 | | ΔeGFR Q5 | |
| (−35.2, −7.8 mL/ | (−7.8, −3.1 mL/ | (−3.1, 0.7 mL/ | ΔeGFR Q4 (0.7, | (5.8, 36.3 mL/ | |
| min/1.73 m²) | min/1.73 m²) | min/1.73 m²) | 5.8 mL/min/1.73 m²) | min/1.73 m²) | P |

Intensive BP Arm (1 y vs BL)

| | | | | | | |
|---|---|---|---|---|---|---|
| ACR^{a} | n = 84; ↓39% (↓51%, | n = 96; ↓32% (↓44%, | n = 84; ↓26% (↓40%, | n = 92; ↓4% (↓21%, | n = 89;↑11% (↓9%, | <0.001 |
| | ↓26%) | ↓18%) | ↓10%) | ↑17%) | ↑35%) | |
| B2M | n = 82; ↓29% (↓51%, | n = 92; ↓3% (↓28%, | n = 82; ↑4% (↓27%, | n = 89; ↑26% (↓11%, | n = 85; ↑45% (↑1%, | 0.07 |
| | ↑2%) | ↑46%) | ↑49%) | ↑80%) | ↑106%) | |
| A1M | n = 88; ↓47% (↓59%, | n = 99; ↓33% (↓48%, | n = 87; ↓17% (↓35%, | n = 96; ↓9% (↓29%, | n = 93; ↓12% (↓31%, | 0.01 |
| | ↓32%) | ↓15%) | ↑6%) | ↑16%) | ↑13%) | |
| IL-18, | n = 94; ↓10% (↓22%, | n = 100; ↓17% (↓28%, | n = 92; ↑15% (↓1%, | n = 99; ↑8% (↓7%, | n = 96; ↑1% (↓13%, | 0.02 |
| ratio | ↑5%) | ↓3%) | ↑33%) | ↑25%) | ↑18%) | |

Standard BP Arm (1 y vs BL)

| | | | | | | |
|---|---|---|---|---|---|---|
| ACR^{a} | n = 35; ↓23% (↓44%, | n = 60; ↓18% (↓36%, | n = 86; ↑14% (↓7%, | n = 111; ↑21% (↑1%, | n = 98; ↑74% (↑43%, | <0.001 |
| | ↑7%) | ↑5%) | ↑41%) | ↑46%) | ↑112%) | |
| B2M | n = 33; ↑13% (↓36%, | n = 61; ↑9% (↓28%, | n = 84; ↑40% (↓2%, | n = 109; ↑43% (↑5%, | n = 98; ↑77% (↑27%, | 0.4 |
| | ↑97%) | ↑64%) | ↑100%) | ↑95%) | ↑145%) | |
| A1M | n = 33; ↑15% (↓21%, | n = 65; ↑9% (↓16%, | n = 87; ↓13% (↓30%, | n = 115; ↔0% (↓28%, | n = 102; ↑19% (↓4%, | 0.4 |
| | ↑65%) | ↑41%) | ↑10%) | ↑21%) | ↑47%) | |
| IL-18 | n = 36; ↑9% (↓13%, | n = 66; ↓8% (↓22%, | n = 91; ↑19% (↑4%, | n = 119; ↑27% (↑13%, | n = 103; ↑36% (↑19%, | 0.005 |
| | ↑36%) | ↑8%) | ↑37%) | ↑44%) | ↑55%) | |

Intensive BP Arm (4 y vs BL)

| | | | | | | |
|---|---|---|---|---|---|---|
| ACR^{a} | n = 93; ↓6% (↓24%, | n = 99; ↓6% (↓24%, | n = 87; ↓14% (↓9%, | n = 96; ↑41% (↑14%, | n = 91; ↑30% (↑4%, | 0.03 |
| | ↑17%) | ↑17%) | ↑43%) | ↑75%) | ↑62%) | |
| B2M | n = 79; ↑15% (↓24%, | n = 83; ↑34% (↓10%, | n = 78; ↑49% (↓1%, | n = 83; ↑19% (↓20%, | n = 81; ↑54% (↑3%, | 0.8 |
| | ↑72%) | ↑100%) | ↑123%) | ↑78%) | ↑130%) | |
| A1M | n = 83; ↓18% (↓39%, | n = 88; ↓26% (↓44%, | n = 83; ↓2% (↓27%, | n = 90; ↓22% (↓41%, | n = 90; ↓14% (↓35%, | 0.7 |
| | ↑9%) | ↓3%) | ↑30%) | ↑3%) | ↑14%) | |
| IL-18 | n = 89; ↑4% (↓12%, | n = 94; ↓5% (↓19%, | n = 86; ↑20% (↑1%, | n = 94; ↑5% (↓11%, | n = 90; ↑3% (↓13%, | 0.4 |
| | ↑23%) | ↑12%) | ↑42%) | ↑24%) | ↑22%) | |

Standand BP Arm (4 y vs BL)

| | | | | | | |
|---|---|---|---|---|---|---|
| ACR^{a} | n = 35; ↑29% (↓11%, | n = 66; ↑82% (↑40%, | n = 90; ↑66% (↑32%, | n = 115; ↑54% (↑26%, | n = 101; ↑81% (↑46%, | 0.5 |
| | ↑86%) | (↑37%) | ↑109%) | ↑88%) | ↑124%) | |
| B2M | n = 30; ↑56% (↓15%, | n = 60; ↑98% (↑30%, | n = 81; ↑57% (↑9%, | n = 103; ↑41% (↑2%, | n = 96; ↑47% (↑5%, | 0.8 |
| | ↑185%) | ↑202%) | ↑127%) | ↑95%) | ↑105%) | |
| A1M | n = 31; ↑28% (↓17%, | n = 60; ↓2% (↓28%, | n = 83; ↓19% (↓38%, | n = 108; ↓7% (↓26%, | n = 101; ↑12% (↑30%, | 0.5 |
| | ↑97%) | ↑34%) | ↑6%) | ↑17%) | ↑12%) | |
| IL-18 | n = 33; ↓12% (↓32%, | n = 61; ↓7% (↓22%, | n = 88; ↑12% (↓4%, | n = 112; ↑3% (↓11%, | n = 101; ↑7% (↓8%, | 0.4 |
| | ↑13%) | ↑12%) | ↑31%) | ↑18%) | ↑23%) | |

Note:
Values are given as number of patients; ratio of 1- or 4-year value to BL (95% CI). Biomarkers with significant ratio of intensive arm to standard arm least-squares means are presented in this table. All urinary biomarker values were log2 transformed and adjusted for linear and quadratic urine creatinine concentrations.
Abbreviations: A1M, α1-microglobulin; ACR, albumin-creatinine ratio; B2M, β2-microglobulin; BL, baseline; BP, blood pressure; CI, confidence interval; CKD, chronic kidney disease; eGFR, estimated glomerular filtration rate; IL-18, interleukin 18; Q, quintile; SD, standard deviation; SPRINT, Systolic Blood Pressure Intervention Trial.
^{a}Model for ACR does not include urine creatinine as covariate.

Next, the sample was limited to participants randomly assigned to the intensive arm and compared the relationship of the magnitude of achieved change in SBP during the trial with concurrent changes in urinary tubule marker levels. At year 1, participants in the intensive arm who achieved the largest SBP reductions (SBP decline>30 mm Hg from baseline) also experienced the greatest reductions in eGFRs (of 11%) and urinary ACRs (of 40%), whereas those with the smallest changes in SBPs experienced the least changes or improvements in eGFRs and urinary ACRs during follow-up (P<0.001). Similarly, a 30% decrease in urinary B2M and 41% decrease in urinary A1M levels among participants with the largest SBP reductions at year 1 and the least changes in those with smaller reductions in SBP (Table 6) was also observed. These observations were no longer apparent at year 4. In comparison, no consistent changes in urinary tubule marker levels across the range of achieved SBP reduction for any of the remaining markers at year 1 was observed (Table 7).

A total of 62 deaths occurred in the experimental sample during a 4-year follow-up: 28 in the intensive BP arm and 34 in the standard BP arm. There were no significant between-group differences for the need for hemodialysis (5 in the intensive BP arm and 7 in the standard BP arm). Two participants were lost during follow-up in the intensive BP arm and none were lost in the standard BP arm.

TABLE 6
Comparison of 1- and 4-Year Changes in eGFR, Albuminuria, and Urinary Tubule Biomarkers Stratified by Quintile
of Systolic Blood Pressure Change Among Participants With CKD Randomly Assigned to the Intensive Treatment Arm

| | | | | | |
|---|---|---|---|---|---|
| ΔSBP Q1 | ΔSBP Q2 | ΔSBP Q3 | ΔSBP Q4 | ΔSBP Q5 | |
| (−66, −30 mm Hg) | (−30, −20 mm Hg) | (−20, −11 mm Hg) | (−11, −0.5 mm Hg) | (−0.5, 55 mm Hg) | P |

Intensive BP Arm (1 y vs BL)

| | | | | | | |
|---|---|---|---|---|---|---|
| eGFR | n = 98; ↓11% ↓14%, | n = 100; ↓7% (↓11%, | n = 102; ↓4% (↓8%, | n = 104; ↓3% (↓7%, | n = 102; ↑6% (↑1%, | <0.001 |
| | ↓7%) | ↓3%) | ↔0%) | ↑1%) | ↑10%) | |
| ACR^{a} | n = 83; ↓40% (↓51%, | n = 87; ↓24% (↓38%, | n = 88; ↓29% (↓41%, | n = 92; ↓23% (↓37%, | n = 91; ↑24% (↑3%, | <0.001 |
| | ↓27%) | ↓7%) | ↓14%) | ↓7%) | ↑50%) | |
| B2M | n = 82; ↓30% (↓51%, | n = 91; ↓2% (↓32%, | n = 81; ↓5% (↓33%, | n = 89; ↑20% (↓15%, | n = 84; ↑73% (↑21%, | 0.01 |
| | ↔0%) | ↑41%) | ↑34%) | ↑31%) | ↑148%) | |
| A1M | n = 88; ↓41% (↓54%, | n = 98; ↓30% (↓45%, | n = 86; ↓31% (↓46%, | n = 96; ↓30% (↓45%, | n = 92; ↑17% (↓8%, | 0.05 |
| | ↓24%) | ↓10%) | ↓13%) | ↓11%) | ↑50%) | |
| IL-18 | n = 94; ↓6% (↓19%, | n = 99; ↓13% (↓25%, | n = 91; ↑4% (↓11%, | n = 99; ↔0% (↓14%, | n = 94; ↑13% (↓2%, | 0.2 |
| | ↑10%) | ↑1%) | ↓20%) | ↑16%) | ↑32%) | |

Intensive BP Arm (4 y vs BL)

| | | | | | | |
|---|---|---|---|---|---|---|
| eGFR | n = 93; ↓9% (↓14%, | n = 100; ↓13% (↓18%, | n = 97; ↓7% (↓12%, | n = 101; ↓5% (↓10% | n = 96; ↓5% (↓10%, | 0.1 |
| | ↓3%) | ↓8%) | ↓2%) | ↔0%) | ↑1%) | |
| ACR^{a} | n = 86; ↑2% (↓18%, | n = 92; ↑14% (↓8%, | n = 94; ↑12% (↓10%, | n = 100; ↑4% (↓15%, | n = 90; ↑39% (↑12%, | 0.3 |
| | ↑28%) | ↑41%) | ↑38%) | ↑29%) | ↑73%) | |
| B2M | n = 79; ↑35% (↓10%, | n = 82; ↑13% (↓25%, | n = 77; ↑27% (↓17%, | n = 83; ↑10% (↓24%, | n = 79; ↑122% (↑46%, | 0.1 |
| | ↑102%) | ↑68%) | ↑92%) | ↑60%) | ↑235%) | |
| A1M | n = 83; ↓24% (↓42%, | n = 87; ↓27% (↓44%, | n = 82; ↓23% (↓42%, | n = 90; ↓21% (↓39%, | n = 88; ↑19% (↓10%, | 0.09 |
| | ↑1%) | ↓4%) | ↑3%) | ↑3%) | ↑58%) | |
| IL-18 | n = 89; ↑10% (↓8%, | n = 93; ↓2% (↓17%, | n = 85; ↓5% (↓20%, | n = 94; ↓13% (↓4%, | n = 88; ↑14% (↓4%, | 0.5 |
| | ↑30%) | ↑15%) | ↑13%) | ↑32%) | ↑35%) | |

Note:
Values are given as number of patients; ratio of 1- or 4-year value to baseline value (95% CI). Biomarkers with significant ratio of intensive arm to standard arm least-squares means are presented in tins table. All urinary biomarkers were log2 transformed and adjusted for linear and quadratic urine creatinine concentrations.
Abbreviations: A1M, α1-microglobulin; ACR, albumin-creatinine ratio; B2M, β2-microglobulin; BL, baseline; BP, blood pressure; CI, confidence interval; CKD, chronic kidney disease; eGFR, estimated glomerular filtration rate; IL-18, interleukin 18; Q, quintile; SBP, systolic blood pressure; SD, standard deviation.
^{a}Model for ACR does not include urine creatinine as covariate.

### Tubule Injury and Dysfunction Biomarkers for Monitoring Therapeutic Responses

In the SPRINT trial, hypertensive participants with high risk for CVD were randomized to intensive systolic blood pressure (SBP) lowering (target<120 mmHg) vs. a standard SBP target (<140 mmHg). The trial showed that intensive SBP lowering resulted in reductions in a composite CVD endpoint and mortality, but also resulted in more rapid loss of eGFR. This has led to considerable concern that the intensive SBP lowering may increase long-term kidney risk. As shown herein, the acute reduction in eGFR reflected hemodynamic changes rather than intrinsic injury to the kidney, which was shown with the novel panel of kidney tubule biomarkers disclosed herein.

Urine biomarkers of tubule function (β₂-microglobulin [B2M], α₁-microglobulin [A1M]), and uromodulin), injury (interleukin 18, kidney injury molecule 1, and neutrophil gelatinase-associated lipocalin), inflammation (monocyte chemoattractant protein 1), and repair (human cartilage glycoprotein 40) were evaluated at baseline, year 1, and year 4. Biomarkers were indexed to urine creatinine concentration and changes between arms were evaluated using mixed-effects linear models and an intention-to-treat approach. Among participants with CKD in SPRINT, random assignment to the intensive SBP arm did not increase any levels of 8 urine biomarkers of tubule cell damage despite loss of eGFR.

Among a subset of 978 trial participants with eGFR<60 ml/min/1.73 m2, a novel panel of 8 biomarkers of kidney tubule injury and dysfunction was measured at baseline and after 1 year in SPRINT. Some of these markers (urine albumin, β2 microglobulin [β2M] and α1 microglobulin [α1M]) are filtered at the glomerulus, while others are produced within the kidney tissue in response to injury, inflammation, or repair (NGAL, KIM-1, IL-18, MCP-1, and YKL-40). As shown herein, despite the acute decline in eGFR, none of these measures showed significant increases in the intensive vs. the standard arm in SPRINT. Accordingly, intensive SBP lowering did not appear to have caused intrinsic tubule cell injury despite the eGFR decline (FIG. 1). Among the tubule biomarker panel, the 3 measures that are filtered at the glomerulus (urine albumin, β2M, and α1M) were all significantly lower in the intensive blood pressure arm (FIG. 1, bars 2-4), whereas none of the biomarkers that are produced within the kidney tissue were significantly different across arms (FIG. 1; bars 5-10). Accordingly, the acute reduction in eGFR in response to intensive SBP lowering is due to hemodynamic changes rather than intrinsic kidney injury in most patients.

The etiologies for reductions in eGFR are heterogeneous, and clinicians do not currently have biomarker tools to differentiate their causes in clinical practice. Given their concerns for intrinsic kidney injury, clinicians often feel compelled to interrupt beneficial medications, such as anti-hypertensive or anti-retroviral medications, and then rely upon observation time to determine whether the kidney “recovers” based upon the eGFR response. For acute interstitial nephritis, many patients require kidney biopsy for definitive diagnosis, which is invasive, expensive, and carries risks of bleeding. By leveraging the unique pathological signatures provided by individual tubule injury and dysfunction biomarkers, the present disclosure provides a novel panel of kidney tubule measures that can be used in the non-invasive diagnosis of the etiology of eGFR decline, and kidney injury. Even when kidney biopsies are obtained, they are rarely done repeatedly. Thus, observing changes in the disclosed biomarkers longitudinally may ultimately provide a useful tool to monitor changes in kidney health non-invasively in response to drug therapy. These association of kidney tubule biomarkers with CKD onset and progression disclosed herein provided an unprecedented new paradigm in nephrology. Collectively, the disclosed biomarkers of kidney tubule injury and dysfunction hold considerable promise, and provide an opportunity to characterize the health of the kidney more fully.

### Equivalents

The present technology is not to be limited in terms of the particular embodiments described in this application, which are intended as single illustrations of individual aspects of the present technology. Many modifications and variations of this present technology can be made without departing from its spirit and scope, as will be apparent to those skilled in the art. Functionally equivalent methods and apparatuses within the scope of the present technology, in addition to those enumerated herein, will be apparent to those skilled in the art from the foregoing descriptions. Such modifications and variations are intended to fall within the scope of the present technology. It is to be understood that this present technology is not limited to particular methods, reagents, compounds compositions or biological systems, which can, of course, vary. It is also to be understood that the terminology used herein is for the purpose of describing particular embodiments only, and is not intended to be limiting.

The disclosures illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the disclosure claimed.

Thus, it should be understood that the materials, methods, and examples provided here are representative of preferred embodiments, are exemplary, and are not intended as limitations on the scope of the disclosure.

The disclosure has been described broadly and generically herein. Each of the narrower species and sub-generic groupings falling within the present disclosure also form part of the disclosure. This includes the generic description of the disclosure with a proviso or negative limitation removing any subject matter from the genus, regardless of whether or not the excised material is specifically recited herein. In addition, where features or aspects of the present disclosure are described in terms of Markush groups, those skilled in the art will recognize that the present disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

Unless otherwise defined, all technical and scientific terms used herein have the same meaning as commonly understood by one of ordinary skill in the art to which this disclosure belongs.

All patents, patent applications, provisional applications, and publications referred to or cited herein are incorporated by reference in their entirety, including all figures and tables, to the extent they are not inconsistent with the explicit teachings of this specification. In case of conflict, the present specification, including definitions, will control.

### REFERENCES

All references are hereby incorporated by reference in their entireties.

1. Lewington, et al. Age-specific relevance of usual blood pressure to vascular mortality: a meta-analysis of individual data for one million adults in 61 prospective studies. Lancet. 2002; 360(9349):1903-1913.

2. Vasan, et al. Impact of high-normal blood pressure on the risk of cardiovascular disease. N Engl J Med. 2001; 345(18):1291-1297.

3. Zanchetti A. Hypertension-related mortality and morbidity. J Hypertens. 2015; 33(10):1979-1980.

4. James P A, Oparil S, Carter B L, et al. 2014 Evidence-based guideline for the management of high blood pressure in adults: report from the panel members appointed to the Eighth Joint National Committee (JNC 8). JAMA. 2014; 311(5):507-520.

5. Neal, et al. Effects of ACE inhibitors, calcium antagonists, and other blood-pressurelowering drugs: results of prospectively designed overviews of randomised trials. Blood Pressure Lowering Treatment Trialists' Collaboration. Lancet. 2000; 356(9246):1955-1964.

6. Malhotra, et al. Association between more intensive vs less intensive blood pressure lowering and risk of mortality in chronic kidney disease stages 3 to 5: a systematic review and meta-analysis. JAMA Intern Med. 2017; 177(10):1498-1505.

7. Hirsch, et al. Tolerating increases in the serum creatinine following aggressive treatment of chronic kidney disease, hypertension and proteinuria: pre-renal success. Am J Nephrol. 2012; 36(5):430-437.

8. Klahr, et al. The effects of dietary protein restriction and blood-pressure control on the progression of chronic renal disease. Modification of Diet in Renal Disease Study Group. N Engl J Med. 1994; 330(13):877-884.

9. Lv, et al. Effects of intensive blood pressure lowering on the progression of chronic kidney disease: a systematic review and meta-analysis. CMAJ. 2013; 185(11):949-957.

10. Wright, et al. Effect of blood pressure lowering and antihypertensive drug class on progression of hypertensive kidney disease: results from the AASK trial. JAMA. 2002; 288(19):2421-2431.

11. Peralta, et al. Effect of intensive versus usual blood pressure control on kidney function among individuals with prior lacunar stroke: a post hoc analysis of the Secondary Prevention of Small Subcortical Strokes (SPS3) Randomized Trial. Circulation. 2016; 133 (6): 584-591.

12. Schrier, et al. Blood pressure in early autosomal dominant polycystic kidney disease. N Engl J Med. 2014; 371(24):2255-2266.

13. SPRINT Research Group; Wright, et al. A randomized trial of intensive versus standard blood-pressure control. N Engl J Med. 2015; 373(22): 2103-2116.

14. Cheung, et al. Effects of intensive BP control in CKD. J Am Soc Nephrol. 2017; 28(9): 2812-2823.

15. Malhotra R, Siew E D. Biomarkers for the early detection and prognosis of acute kidney injury. Clin J Am Soc Nephrol. 2017; 12(1):149-173.

16. Peralta, et al. Associations of urinary levels of kidney injury molecule 1 (KIM-1) and neutrophil gelatinase-associated lipocalin (NGAL) with kidney function decline in the Multi-Ethnic Study of Atherosclerosis (MESA). Am J Kidney Dis. 2012; 60(6):904-911.

17. Scherzer, et al. Use of urine biomarker-derived clusters to predict the risk of chronic kidney disease and all-cause mortality in HIV-infected women. Nephrol Dial Transplant. 2016; 31(9):1478-1485.

18. Ambrosius, et al. The design and rationale of a multicenter clinical trial comparing two strategies for control of systolic blood pressure: the Systolic Blood Pressure Intervention Trial (SPRINT). Clin Trials. 2014; 11(5):532-546.

19. Jotwani, et al. Association of urine alphal-microglobulin with kidney function decline and mortality in HIV-infected women. Clin J AmSoc Nephrol. 2015; 10(1):63-73.

20. Parikh, et al. Urine NGAL and IL-18 are predictive biomarkers for delayed graft function following kidney transplantation. Am J Transplant. 2006; 6(7): 1639-1645

21. Waikar, et al. Relationship of proximal tubular injury to chronic kidney disease as assessed by urinary kidney injury molecule-1 in five cohort studies. Nephrol Dial Transplant. 2016; 31(9):1460-1470.

22. Park, et al. Urinary markers of fibrosis and risk of cardiovascular events and death in kidney transplant recipients: the FAVORIT Trial. Am J Transplant. 2017; 17(10): 2640-2649.

23. Schmidt, et al. Chitinase-like protein Brp-39/YKL-40 modulates the renal response to ischemic injury and predicts delayed allograft function. J Am Soc Nephrol. 2013; 24(2):309-319.

24. Ix, et al. Urine fibrosis markers and risk of allograft failure in kidney transplant recipients: a case-cohort ancillary study of the FAVORIT Trial. Am J Kidney Dis. 2017; 69(3):410-419.

25. Nadkarni, et al. Association of urinary biomarkers of inflammation, injury, and fibrosis with renal function decline: the ACCORD Trial. Clin J Am Soc Nephrol. 2016; 11(8):1343-1352.

26. Hall, et al. IL-18 and urinary NGAL predict dialysis and graft recovery after kidney transplantation. J Am Soc Nephrol. 2010; 21(1):189-197.

27. Puthumana, et al. YKL-40 associates with renal recovery in deceased donor kidney transplantation. J Am Soc Nephrol. 2017; 28(2):661-670.

28. Cherney, et al. Renal hemodynamic effect of sodium-glucose cotransporter 2 inhibition in patients with type 1 diabetes mellitus. Circulation. 2014; 129(5): 587-597.

29. Tang, et al. Sodium-glucose cotransporter 2 inhibitors and risk of adverse renal outcomes among type 2 diabetes patients: a network and cumulative meta-analysis of randomized controlled trials. Diabetes Obes Metab. 2017; 19(8):1106-1115.

30. Wanner, et al. Empagliflozin and progression of kidney disease in type 2 diabetes. N Engl J Med. 2016; 375(18):323-334.

31. Nadkarni, et al. Effect of intensive blood pressure lowering on kidney tubule injury: findings from the ACCORD Trial study participants. Am J Kidney Dis. 2018; doi:10.1053/j.ajkd.2018.07.016.

32. Ong H T, Ong L M, Ho J J. Angiotensin-Converting Enzyme Inhibitors (ACEIs) and Angiotensin-Receptor Blockers (ARBs) in Patients at High Risk of Cardiovascular Events: A Meta-Analysis of 10 Randomised Placebo-Controlled Trials. ISRN Cardiol. 2013; 2013:478597.

33. Nath K A. Tubulointerstitial changes as a major determinant in the progression of renal damage. Am J Kidney Dis Off J Natl Kidney Found. 1992 July; 20(1):1-17.

34. Rule A D, Amer H, Cornell L D, Taler S J, Cosio F G, Kremers W K, et al. The association between age and nephrosclerosis on renal biopsy among healthy adults. Ann Intern Med. 2010 May 4; 152(9):561-7.

35. Howie A J, Ferreira M A, Adu D. Prognostic value of simple measurement of chronic damage in renal biopsy specimens. Nephrol Dial Transplant Off Publ Eur Dial Transpl Assoc—Eur Ren Assoc. 2001 June; 16(6):1163-9.

36. Takebayashi S, Kiyoshi Y, Hisano S, Uesugi N, Sasatomi Y, Meng J, et al. Benign nephrosclerosis: incidence, morphology and prognosis. Clin Nephrol. 2001 May; 55(5):349-56.

37. American Diabetes Association, Treatment of Hypertension in Adults With Diabetes, Diabetes Care 25(suppl 1): s71-s73 (2002).

38. Yang, R., Smolders, I. & Dupont, A. Blood pressure and renal hemodynamic effects of angiotensin fragments. Hypertens Res 34, 674-683 (2011).

TABLE 5
Comparison of 1-Year and 4-Year Changes in Urinary Tubule Biomarkers## Stratified
by Quintile of Estimated GFR Change among Participants with CKD in SPRINT, Stratified by Treatment Arm.

| | | | | | | |
|---|---|---|---|---|---|---|
| | QI (Largest | | | | Q5 | |
| Quintile of | eGFR decline) | Q2 | Q3 | Q4 | (Largest eGFR increase) | |
| Δ eGFR | (−35.2, −7.8) | (−7.8, −3.1) | (−3.1, 0.7) | (0.7, 5.8) | (5.8, 36.3) | |
| (1 year) | (mL/min/1.73 m²) | (mL/min/1.73 m²) | \ (mL/min/1.73 m²) | (mL/min/1.73 m²) | (mL/min/1.73 m²) | P-value |

Intensive BP Arm (Ratio of 1 year to Baseline)

| | | | | | | |
|---|---|---|---|---|---|---|
| Total n | 94 | 100 | 92 | 99 | 96 | |
| uYKL-40, ratio | ↓31% (↓49%, | ↓10% (↓34%, | ↓ 3% (↓29%, | ↑24% (↓ 9%, | ↑34% (↓ 2%, | 0.02 |
| (95% CI) | ↓ 7%) | ↑22%) | ↑32%) | ↑68%) | ↑81%) | |
| Total n | 88 | 98 | 87 | 96 | 92 | |
| uUMOD, ratio | ↓14% (↓24%, | ↓12% (↓21%, | ↓ 7% (↓17%, | ↓ 2% (↓13%, | ↑14% (↑ 1%, | 0.01 |
| (95% CI) | ↓ 4%) | ↓ 1%) | ↑ 4%) | ↑ 9%) | ↑27%) | |
| Total n | 94 | 100 | 92 | 99 | 96 | |
| uMCP-1, ratio | ↑12% (↓ 4%, | ↓ 7% (↓20%, | ↓ 4% (↓18%, | ↓12% (↓25%, | ↓24% (↓35%, | 0.01 |
| (95% CI) | ↑31%) | ↑ 9%) | ↑12%) | ↑ 2%) | ↓11%) | |
| Total n | 94 | 100 | 92 | 99 | 96 | |
| uKIM-1, ratio | ↓16% (↓23%, | ↓ 8% (↓21%, | ↑ 4% (↓10%, | ↑ 5% (↓ 9%, | ↓10% (↓22%, | 0.2 |
| (95% CI) | ↑ 3%) | ↑ 6%) | ↑20%) | ↑21%) | ↑ 4%) | |
| Total n | 88 | 99 | 87 | 96 | 93 | |
| uNGAL, ratio | ↑22% (↓ 1%, | ↓ 2% (↓20%, | ↓ 3% (↓21%, | ↑19% (↓ 3%, | ↑ 1% (↓13%, | 0.4 |
| (95% CI) | ↑50%) | ↑20%) | ↑20%) | ↑47%) | ↑18%) | |

Standard BP Arm (Ratio of 1 year to Baseline)

| | | | | | | |
|---|---|---|---|---|---|---|
| Total n | 36 | 66 | 91 | 119 | 103 | |
| uYKL-40, ratio | ↑37% (↓12%, | ↑ 6% (↓23%, | ↑15% (↓13%, | ↑ 2% (↓20%, | ↑56% (↑20%, | 0.2 |
| (95% CI) | ↑113%) | ↑46%) | ↑52%) | ↑30%) | ↑103%) | |
| Total n | 33 | 65 | 87 | 115 | 102 | |
| uUMOD, ratio | ↓26% (↓41%, | ↓ 5% (↓19%, | ↑ 7% (↓ 6%, | ↑19% (↑ 6%, | ↑21% (↑ 7%, | <0.001 |
| (95% CI) | ↓ 9%) | ↑11%) | ↑23%) | ↑34%) | ↑37%) | |
| Total n | 36 | 66 | 91 | 119 | 103 | |
| uMCP-1, ratio | ↑43% (↑16%, | ↑16% (↔0%, | ↑17% (↑ 3%, | ↑ 8% (↓ 4%, | −0% (↓12%, | 0.04 |
| (95% CI) | ↑76%) | ↑35%) | ↑34%) | ↑21%) | ↑13%) | |
| Total n | 36 | 66 | 91 | 119 | 103 | |
| uKIM-1, ratio | ↑ 6% (↓16%, | ↑14% (↓ 4%, | ↑30% (↑13%, | ↑23% (↑ 8%, | ↑17% (↑ 2%, | 0.6 |
| (95% CI) | ↑34%) | ↑34%) | ↑50%) | ↑39%) | ↑34%) | |
| Total n | 33 | 65 | 87 | 115 | 102 | |
| uNGAL, ratio | ↑42% (↑ 6%, | ↑10% (↓11%, | ↑14% (↓ 5%, | ↑11% (↓ 6%, | ↑22% (↑ 3%, | 0.6 |
| (95% CI) | ↑90%) | ↑36%) | ↑36%) | ↑29%) | ↑45%) | |

Intensive BP Arm (Ratio of 4 year to Baseline)

| | | | | | | |
|---|---|---|---|---|---|---|
| Total n | 89 | 94 | 86 | 94 | 90 | |
| uYKL-40, ratio | ↑22% (↓15%, | ↑27% (↓11%, | ↑24% (↓15%, | ↑23% (↓14%, | ↑28% (↓12%, | 0.1 |
| (95% CI) | ↑77%) | ↑83%) | ↑80%) | ↑77%) | ↑15%) | |
| Total n | 83 | 87 | 83 | 90 | 90 | |
| uUMOD, ratio | ↓30% (↓40%, | ↓28% (↓37%, | ↓27% (↓37%, | ↓24% (↓34%, | ↓12% (↓23%, | 0.2 |
| (95% CI) | ↓19%) | ↓16%) | ↓16%) | ↓13%) | ↑ 2%) | |
| Total n | 89 | 94 | 86 | 94 | 90 | |
| uMCP-1, ratio | ↓12% (↓25%, | ↑10% (↓23%, | ↑ 8% (↓ 8%, | ↓16% (↓28%, | ↓4% (↓18%, | 0.2 |
| (95% CI) | ↑ 3%) | ↑ 5%) | ↑27%) | ↓ 1%) | ↑12%) | |
| Total n | 89 | 94 | 86 | 94 | 90 | |
| uKIM-1, ratio | ↓ 4% (↓17%, | ↑ 6% (↓ 8%, | ↑36% (↓18%, | ↑ 1%(↓12%, | ↑11% (↓ 4%, | 0.01 |
| (95% CI) | ↑10%) | ↑21%) | ↑57%) | ↑16%) | ↑27%) | |
| Total n | 88 | 98 | 86 | 96 | 92 | |
| uNGAL, ratio | ↓ 3% (↓22%, | ↑15% (↓ 7%, | ↑10% (↓11%, | ↓10% (↓27%, | ↑31% (↑ 6%, | 0.1 |
| (95% CI) | ↑20%) | ↑42%) | ↑35%) | ↑10%) | ↑ 61%) | |

Standard BP Arm (Ratio of 4 year to Baseline)

| | | | | | | |
|---|---|---|---|---|---|---|
| Total n | 33 | 61 | 88 | 112 | 101 | |
| uYKL-40, ratio | ↑33% (↓25%, | ↑61% (↑ 6%, | ↑39% (↓ 2%, | ↑39% (↑ 2%, | ↑56% (↑12%, | 0.9 |
| (95% CI) | ↑136%) | ↑146%) | ↑97%) | ↑90%) | ↑116%) | |
| Total n | 31 | 60 | 83 | 108 | 101 | |
| uUMOD, ratio | ↓42% (↓55%, | ↓20% (↓33%, | ↓27% (↓38%, | ↓ 8% (↓20%, | ↓ 4% (↓16%, | 0.005 |
| (95% CI) | ↓25%) | ↓ 4%) | ↓15%) | ↑ 5%) | ↑10%) | |
| Total n | 33 | 61 | 88 | 112 | 101 | |
| uMCP-1, ratio | ↓ 2% (↓25%, | ↓10% (↓26%, | ↓ 2% (↓17%, | ↑ 4% (↓10%, | ↓18% (↓30%, | 0.3 |
| (95% CI) | ↑29%) | ↑10%) | ↑16%) | ↑21%) | ↓ 4%) | |
| Total n | 33 | 61 | 88 | 112 | 101 | |
| uKIM-1, ratio | ↓ 3% (↓26%, | ↑ 7% (↓12%, | ↑14% (↓ 3%, | ↑10% (↓ 5%, | ↑ 7% (↓ 8%, | 0.9 |
| (95% CI) | ↑27%) | ↑30%) | ↑35%) | ↑27%) | ↑25%) | |
| Total n | 31 | 60 | 83 | 108 | 101 | |
| uNGAL, ratio | ↓ 3% (↓33%, | ↑16% (↓11%, | ↑29% (↑ 3%, | ↑ 3% (↓15%, | ↑12% (↓ 9%, | 0.6 |
| (95% CI) | ↑40%) | ↑50%) | ↑61%) | ↑26%) | ↑37%) | |

##Biomarkers with non-significant ratio of intensive arm to standard arm least-squares means are presented in this table. All urine biomarkers were log2 transformed and adjusted for linear and quadratic urine creatinine concentrations.
Abbreviations: SD; standard deviation. SBP; systolic blood pressure, CI; confidence interval, uYKL-40; urinary human cartilage glycoprotein-39, uUMOD; urinary uromodulin, uMCP-1; urinary monocyte chemoattractant protein 1, uKIM-1; urinary' kidney injury molecule-1, uNGAL; neutrophil gelatinase-associated lipocalin.

TABLE 7
Comparison of 1- and 4-year Changes in Urinary Tubule Biomarkers^{##} Stratified by Quintile
of Systolic Blood Pressure Change among Participants with CKD, Randomized to the Intensive Treatment Arm

| | | | | | | |
|---|---|---|---|---|---|---|
| | | | | | Q5 | |
| Quintile of | Q1 | Q2 | Q3 | Q4 | (Largest SBP increase) | |
| Δ SBP | (Largest SBP decline) | (−30, −20) | (−20, −11) | (−11, −0.5) | (−0.5, 55) | |
| (1 year) | (−66, −30) (mm Hg) | (mm Hg) | (mm Hg) | (mm Hg) | (mm Hg) | P-value |

Intensive BP Arm (Ratio of 1 year to Baseline)

| | | | | | | |
|---|---|---|---|---|---|---|
| Total n | 94 | 99 | 91 | 99 | 94 | |
| uYKL-40, ratio | ↓13% (↓37%, | ↑ 2% (↓26%, | ↓15% (↓38%, | ↓ 6% (↓31%, | ↑42% (↑ 4%, | 0.1 |
| (95% CI) | ↑19%) | ↑39%) | ↑15%) | ↑28%) | ↑ 93%) | |
| Total n | 88 | 97 | 86 | 96 | 91 | |
| uUMOD, ratio | ↓ 6% (↓17%, | ↓10% (↓20%, | ↑ 1% (↓12%, | (↓ 6% (↓16%, | ↓ 4% (↓14%, | 0.8 |
| (95% CI) | ↑ 6%) | ↑ 1%) | ↑11%) | ↑5%) | ↑8%) | |
| Total n | 94 | 99 | 91 | 99 | 94 | |
| uMCP-1, ratio | ↓ 4% (↓18%, | ↓ 9% (↓22%, | ↓ 4% (↓18%, | ↓ 9% (↓22%, | ↓ 9% (↓22%, | 0.9 |
| (95% CI) | ↑13%) | ↑ 6%) | ↑12%) | ↑ 6%) | ↑ 6%) | |
| Total n | 94 | 99 | 91 | 99 | 94 | |
| uKIM-1, ratio | ↓15% (↓27%, | ↓ 4% (↓18%, | ↑ 3% (↓11%, | (↓11% (↓23%, | ↑ 4% (↓10%, | 0.2 |
| (95% CI) | ↓2%) | ↑11%) | ↑19%) | ↑2%) | ↑ 20%) | |
| Total n | 88 | 98 | 86 | 96 | 92 | |
| uNGAL, ratio | ↓ 3% (↓22%, | ↑15% (↓ 7%, | ↑10% (↓11%, | (↓10% (↓27%, | ↑31% (↓ 6%, | 0.1 |
| (95% CI) | ↑20%) | ↑42%) | ↑35%) | ↑10%) | ↓ 61%) | |

Intensive BP Arm (Ratio of 4 year to Baseline)

| | | | | | | |
|---|---|---|---|---|---|---|
| Total n | 89 | 93 | 85 | 94 | 88 | |
| uYKL-40, ratio | ↑ 5% (↓28%, | ↑43% (↔0%, | ↓ 3% (↓33%, | ↑17% (↓17%, | ↑79% (↑23%, | 0.2 |
| (95% CI) | ↑51%) | ↑103%) | ↑41%) | ↑66%) | ↑160%) | |
| Total n | 83 | 86 | 92 | 90 | 88 | |
| uUMOD, ratio | ↓24% (↓34%, | ↓28% (↓38%, | ↓25% (↓35%, | ↓26% (↓36%, | ↓20% (↓31%, | 0.9 |
| (95% CI) | ↓12%) | ↓18%) | ↓12%) | ↓15%) | ↓ 7%) | |
| Total n | 89 | 93 | 85 | 94 | 88 | |
| uMCP-1, ratio | ↓21% (↓33%, | ↑ 6% (↓ 9%, | ↓15% (↓28%, | ↑ 1% (↓13%, | ↓ 4% (↓18%, | 0.05 |
| (95% CI) | ↓7%) | ↑24%) | ↔0%) | ↑17%) | ↑13%) | |
| Total n | 89 | 93 | 85 | 94 | 88 | |
| uKIM-1, ratio | ↓ 6% (↓18%, | ↑17% (↑ 2%, | ↑ 9% (↓ 6%, | ↑11% (↓ 3%, | ↑15% (↓ 1%, | 0.3 |
| (95% CI) | ↑ 9%) | ↑34%) | ↑26%) | ↑27%) | ↑33%) | |
| Total n | 83 | 87 | 82 | 90 | 88 | |
| uNGAL, ratio | ↓ 3% (↓24%, | ↑25% (↔0%, | ↑ 2% (↓20%, | ↑ 9% (↓13%, | ↑46% (↑15%, | 0.1 |
| (95% CI) | ↑23%) | ↑57%) | ↑31%) | ↑36%) | ↑85%) | |

^{##}Biomarkers with non-significant ratio of intensive arm to standard arm least-squares means are presented in this table. All urine biomarkers were log2 transformed and adjusted for linear and quadratic urine creatinine concentrations.
Abbreviations: SD; standard deviation. SBP; systolic blood pressure, CI; confidence interval, uYKL-40; urinary human cartilage glycoprotein-39, uUMOD; urinary uromodulin, uMCP-1; urinary monocyte chemoattractant protein 1, uKIM-1; urinary kidney injury molecule-1, uNGAL; neutrophil gelatinase-associated lipocalin.

What is claimed is:

1. A method for treating diabetes mellitus in a subject in need thereof comprising:
(a) administering a therapeutic regimen to the subject after the levels of one or more of: α₁-microglobulin (A1M), β₂-microglobulin (B2M), kidney injury molecule 1 (KIM-1), interleukin 18 (IL-18), monocyte chemoattractant protein 1 (MCP-1), neutrophil gelatinase-associated lipocalin (NGAL), uromodulin (UMOD), or human cartilage glycoprotein 39 (YKL-40) were measured in a first biological sample isolated from the subject; and
(b) comparing the levels of the one or more of: A1M, B2M, KIM-1, IL-18, MCP-1, NGAL, UMOD, and YKL-40 measured in a second biological sample from the subject to the measured levels in the first biological sample.

2. The method of claim 1, wherein the measured levels of the one or more of comprise the levels of KIM-1, IL-18, MCP-1, NGAL, UMOD, and YKL-40.

3. The method of claim 1, wherein the measured levels of the one or more comprise the levels of selected from KIM-1, NGAL and UMOD; MCP-1, IL-18 and YKL-40; UMOD, MCP-1, and IL-18; NGAL, MCP-1, and IL-18; KIM-1, MCP-1 and IL-18.

4. The method of claim 1, wherein the measured levels of the one or more levels comprise:
(1) the levels of at least one of UMOD, NGAL, KIM-1, and (2) the levels of at least one of MCP-1, IL-18 or YKL-40.

5. The method of claim 1, further comprising repeating step (a)-(b) during treatment.

6. The method of claim 1, wherein the first and second biological samples are blood, urine or both. The method of claim 1, further comprising administering the therapeutic regimen to the subject if the measured levels of the one or more of: KIM-1, IL-18, MCP-1, NGAL, UMOD, or YKL-40 in the second biological sample are not elevated when compared to the measured levels in the first biological sample.

8. The method of claim 1, wherein a lack of elevated levels of the one or more of: KIM-1, IL-18, MCP-1, NGAL, UMOD, or YKL-40 in the second biological sample as compared to the measured levels of the first biological sample is indicative of continued kidney health and the therapeutic regimen should be continued.

9. The method of claim 1, wherein the therapeutic regimen comprises administering a therapeutic selected from sodium glucose transporter 2 (SGLT2) inhibitors, angiotensin-converting enzymes inhibitors, nonsteroidal anti-inflammatory medications, antihypertensive medications, intensive blood pressure lowering medications or a combination thereof.

10. The method of claim 1, wherein the therapeutic regimen comprises administering at least one sodium glucose transporter 2 (SGLT2) inhibitor selected from or canagliflozin, dapagliflozin, and empagliflozin.

11. The method of claim 7, further comprising administering a non- SGLT2 inhibitor therapeutic regimen and wherein the non-SGLT2 inhibitor therapeutic for the treatment of diabetes mellitus is selected from metformin, sulphonylureas, nateglinide, repaglinide, thiazolidinediones, pioglitazone PPARa-glucosidase inhibitors, insulin and insulin analogues, Glucagon-like peptide 1 (GLP-1) and GLP-1 analogues or dipeptidyl peptidase-4 (DPP-4) inhibitors.

12. The method of claim 1, wherein the therapeutic regimen comprises administering an intensive blood pressure lowering therapy.

13. The method of claim 12, wherein the intensive blood pressure lowering therapy is an antihypertensive regimen selected from diuretics, renin-angiotensin system (RAS) antagonists, β-adrenergic blockers, a-adrenergic blockers, calcium channel blockers, or a combination thereof.

14. The method of claim 13, wherein the antihypertensive regimen is selected from chlorthalidone, chlorothiazide, hydrochlorothiazide, indapamide, and metolazone, furosemide, bumetanide, amlodipine, Azilsartan, or acebutolol.

15. The method of claim 1, wherein the subject is at risk of an adverse health condition when the estimated glomerular filtration rates (eGFR) of the subject is less than 60 ml/min/1.73 m², and wherein the eGFR is measured in the first and second biological samples.

16. The method of claim 15, wherein the therapeutic regiment is discontinued if the levels of the one or more of: KIM-1, IL-18, MCP-1, NGAL, UMOD, or YKL-40 in the second biological sample are elevated when compared to the measured levels of the first biological sample in conjunction with a rapid loss of eGFR, A1M, or B2M.

17. The method of claim 1, wherein the loss of eGFR is at least 11% reduction, or eGFR in the second biological sample is less than 40 ml/min/1.73 m².

18. The method of claim 1, wherein the subject is at risk of renal impairment or chronic kidney disease.

19. The method of claim 1, further comprising discontinuing the therapeutic regiment if the measured levels of the one or more of: KIM-1, IL-18, MCP-1, NGAL, UMOD, or YKL-40 in the second biological sample are elevated when compared to the measured levels in the first biological sample, and the eGFR is reduced by at least 11%, or the eGFR in the second biological sample is less than 40 ml/min/1.73 m².

20. A method of treating hypertension in a subject at risk of chronic kidney disease comprising:
(a) administering a therapeutic regimen to the subject after the level of one of α₁-microglobulin (A1M), β₂-microglobulin (B2M), kidney injury molecule 1 (KIM-1), interleukin 18 (IL-18), monocyte chemoattractant protein 1 (MCP-1), neutrophil gelatinase-associated lipocalin (NGAL), uromodulin (UMOD), or human cartilage glycoprotein 39 (YKL-40) were measured in a first biological sample isolated from the subject; and
(b) comparing the measured levels of the one or more of A1M, B2M, KIM-1, IL-18, MCP-1, NGAL, UMOD, or YKL-40 in a second biological sample from the subject to the measured level of the first biological sample.

21. The method of claim 20, wherein the measured levels of the one or more comprise the levels of KIM-1, IL-18, MCP-1, NGAL, UMOD, and YKL-40.

22. The method of claim 20, wherein the measured levels of the one or more comprise the levels selected from KIM-1, NGAL and UMOD; MCP-1, IL-18 and YKL-40; UMOD, MCP-1, and IL-18; NGAL, MCP-1, and IL-18; KIM-1, MCP-1 and IL-18.

23. The method of claim 20, wherein the measured levels of the one or more comprise: (1) the levels of at least one of UMOD, NGAL, KIM-1, and (2) the levels of at least one of MCP-1, IL-18 or YKL-40.

24. The method of claim 20, further comprising administering the therapeutic regimen to the subject if the measured levels the one or more of KIM-1, IL-18, MCP-1, NGAL, UMOD, and YKL-40 in the second biological sample are not elevated when compared to the measured level in the first biological sample.

25. The method of claim 20, wherein a lack of elevated measured levels of the one or more of KIM-1, IL-18, MCP-1, NGAL, UMOD, or YKL-40 in the second biological sample as compared to the measured levels of the first biological sample is indicative of continued kidney health and the therapeutic regimen should be continued.

26. The method of claim 20, wherein the first and second biological samples are blood, urine or both.

27. The method of claim 20, further comprising repeating step (a)-(b) during treatment.

28. The method of claim 20, wherein the therapeutic regimen is an antihypertensive regimen selected from diuretics, renin-angiotensin system (RAS) antagonists, β-adrenergic blockers, α-adrenergic blockers, calcium channel blockers, or a combination thereof.

29. The method of claim 28, wherein the antihypertensive regimen is selected chlorthalidone, chlorothiazide, hydrochlorothiazide, indapamide, and metolazone, furosemide, bumetanide, amlodipine, Azilsartan, or acebutolol.

30. The method of claim 20, wherein the therapeutic regiment is discontinued if the measured levels of KIM-1, IL-18, MCP-1, NGAL, UMOD, or YKL-40 in the second biological sample are elevated when compared to the measured levels of the first biological sample in conjunction with a rapid loss of eGFR, A1M, or B2M.

31. The method of claim 24, wherein the loss of eGFR is at least 11% reduction, or eGFR in the second biological sample is less than 40 ml/min/1.73 m².

32. A kit or article of manufacture comprising: (i) reagents specific to measure the levels of one or more of α₁-microglobulin (A1M), β₂-microglobulin (B2M), kidney injury molecule 1 (KIM-1), interleukin 18 (IL-18), monocyte chemoattractant protein 1 (MCP-1), neutrophil gelatinase-associated lipocalin (NGAL), uromodulin (UMOD), or human cartilage glycoprotein 39 (YKL-40) in a biological sample from a subject; and (ii) instructions for monitoring kidney health in the subject undergoing treatment for a therapeutic regimen that induces renal impairment or chronic kidney disease.
